SEQUENCE LISTING (22 sequences)

SEQ ID NO: 1 - protein, 4 aa, Artificial sequence
  peptide
KRSS

SEQ ID NO: 2 - protein, 5 aa, Artificial sequence
  peptide
PGESS

SEQ ID NO: 3 - protein, 4 aa, Artificial sequence
  peptide
SLTQ

SEQ ID NO: 4 - protein, 4 aa, Artificial sequence
  peptide
AMGN

SEQ ID NO: 5 - protein, 5 aa, Artificial sequence
  peptide
GDRLV

SEQ ID NO: 6 - protein, 7 aa, Artificial sequence
  peptide
RGGKRSS

SEQ ID NO: 7 - protein, 7 aa, Artificial sequence
  peptide
SLTQDQG

SEQ ID NO: 8 - protein, 7 aa, Artificial sequence
  peptide
ADAQADV

SEQ ID NO: 9 - protein, 7 aa, Artificial sequence
  peptide
RAKGRDA

SEQ ID NO: 10 - protein, 7 aa, Artificial sequence
  peptide
KASRLGR

SEQ ID NO: 11 - protein, 7 aa, Artificial sequence
  peptide
RGASMDG

SEQ ID NO: 12 - protein, 7 aa, Artificial sequence
  peptide
GRPGESS

SEQ ID NO: 13 - protein, 7 aa, Artificial sequence
  peptide
SRTEGDV

SEQ ID NO: 14 - protein, 9 aa, Artificial sequence
  peptide
CRGGKRSSC

SEQ ID NO: 15 - protein, 9 aa, Artificial sequence
  peptide
CSLTQDQGC

SEQ ID NO: 16 - protein, 9 aa, Artificial sequence
  peptide
CADAQADVC

SEQ ID NO: 17 - protein, 9 aa, Artificial sequence
  peptide
CRAKGRDAC

SEQ ID NO: 18 - protein, 9 aa, Artificial sequence
  peptide
CKASRLGRC

SEQ ID NO: 19 - protein, 9 aa, Artificial sequence
  peptide
CRGASMDGC

SEQ ID NO: 20 - protein, 9 aa, Artificial sequence
  peptide
CGRPGESSC

SEQ ID NO: 21 - protein, 9 aa, Artificial sequence
  peptide
CSRTEGDVC

SEQ ID NO: 22 - protein, 9 aa, Artificial sequence
  peptide
CSGRRGKSC

(19) United States
(12) Patent Application Publication
(10) Pub. No.: US 2024/0058469 A1
(43) Pub. Date: Feb. 22, 2024

(54) USE OF PSILOCYBIN IN CANCER TREATMENT

(71) Applicant: Silo Pharma, Inc., Englewood Cliffs, NJ (US)

(72) Inventor: Eric Weisblum, Englewood Cliffs, NJ (US)

(21) Appl. No.: 18/257,014

(22) PCT Filed: Dec. 10, 2021
(86) PCT No.: PCT/US2021/062888

§ 371 Date: Jun. 12, 2023

Related U.S. Application Data
(60) Provisional application No. 63/124,827, filed on Dec. 13, 2020.

(51) Int. Cl. A61K 47/69; A61K 45/06; A61K 31/675; A61K 9/00; C07K 14/47

(52) U.S. Cl. CPC A61K 47/6911; A61K 45/06; A61K 31/675; A61K 9/0019; C07K 14/47

(57) ABSTRACT

Disclosed herein include methods, compositions, and kits for treating or alleviating one or more symptoms of a proliferative disease, including cancer. In some embodiments, a composition for use in treating or alleviating one or more symptoms of the proliferative disease (for example cancer) comprises a targeting peptide associated with psilocybin or an analog thereof.

### CROSS-REFERENCE TO RELATED APPLICATIONS

This application is a U.S. national phase application under 35 U.S.C. § 371 of International Application No. PCT/US2021/062888, filed on Dec. 10, 2021, which claims the benefit under 35 U.S.C. § 119(e) to U.S. Provisional Patent Application No. 63/124,827 filed on Dec. 13, 2020. The content of this related applications is incorporated herein by reference in its entirety for all purposes.

### REFERENCE TO SEQUENCE LISTING

The present application is being filed along with a Sequence Listing in electronic format. The Sequence Listing is provided as a file entitled Sequence Listing, created Nov. 14, 2021, which is 4 kilobytes in size. The information in the electronic format of the Sequence Listing is incorporated herein by reference in its entirety.

### BACKGROUND

### Field

The present disclosure relates generally to the field of disease treatment, for example targeted treatment for proliferative diseases including cancer.

### Description of the Related Art

Targeted delivery of a drug, prodrug, or therapeutic agent to cells that cause a disease or are affected by a disease can improve treatment of the disease. There is a need for targeted delivery of drugs, prodrugs, or other therapeutic agents.

### SUMMARY

Disclosed herein include methods of treating a proliferative disease. In some embodiments, the method comprises: administering to a subject in need thereof a therapeutically effective amount of a pharmaceutical composition, where the pharmaceutical composition comprises a targeting peptide comprising an amino acid sequence of any one of SEQ ID NOs: 1-22 associated with psilocybin, or a pharmaceutically acceptable salt, co-crystal, polymorph, hydrate, solvate, stereoisomer, or pro-drug of psilocybin, and where the targeting peptide is capable of delivering the psilocybin, or the pharmaceutically acceptable salt, co-crystal, polymorph, hydrate, solvate, stereoisomer, or pro-drug of psilocybin, to a target environment of the subject.

Disclosed herein also include methods of alleviating one or more symptoms of a proliferative disease, or preventing or delaying the onset of one or more symptoms of a proliferative disease, in a subject in need thereof. The method can comprises administering to the subject a therapeutically effective amount of a pharmaceutical composition, where the pharmaceutical composition comprises a targeting peptide comprising an amino acid sequence of any one of SEQ ID NOs: 1-22 associated with psilocybin, or a pharmaceutically acceptable salt, co-crystal, polymorph, hydrate, solvate, stereoisomer, or pro-drug of psilocybin, and where the targeting peptide is capable of delivering the psilocybin, or the pharmaceutically acceptable salt, co-crystal, polymorph, hydrate, solvate, stereoisomer, or pro-drug of psilocybin, to a target environment of the subject.

The one or more symptoms can comprise pain. The pain can be bone pain, joint pain, back pain, neck pain, pain caused by spinal cord compression, or a combination thereof. In some embodiments, the pain is an acute pain, a chronic pain, or a combination thereof. In some embodiments, the pain is a somatic pain, a neuropathic pain, a visceral pain, or a combination thereof.

The proliferative disease can be cancer, for example carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, or a combination thereof. In some embodiments, the disease is a solid tumor, and optionally the solid tumor is neuroblastoma, Ewing sarcoma or Wilms tumor. In some embodiments, the disease is a liquid tumor.

The targeting peptide can be a central nervous system (CNS) targeting peptide. In some embodiments, the target environment is the nervous system, and optionally the nervous system is the CNS.

In some embodiments, the targeting peptide is directly associated with psilocybin, or a pharmaceutically acceptable salt, co-crystal, polymorph, hydrate, solvate, stereoisomer, or pro-drug of psilocybin. In some embodiments, the targeting peptide is covalently attached with psilocybin, or a pharmaceutically acceptable salt, co-crystal, polymorph, hydrate, solvate, stereoisomer, or pro-drug of psilocybin. In some embodiments, the targeting peptide is covalently attached with psilocybin, or a pharmaceutically acceptable salt, co-crystal, polymorph, hydrate, solvate, stereoisomer, or pro-drug of psilocybin, via a saturated or unsaturated, substituted or unsubstituted, straight or branched carbon chain. In some embodiments, the targeting peptide is conjugated to psilocybin, or a pharmaceutically acceptable salt, co-crystal, polymorph, hydrate, solvate, stereoisomer, or pro-drug of psilocybin. In some embodiments, the targeting peptide is non-covalently attached with psilocybin, or a pharmaceutically acceptable salt, co-crystal, polymorph, hydrate, solvate, stereoisomer, or pro-drug of psilocybin. In some embodiments, the targeting peptide is indirectly associated with psilocybin, or a pharmaceutically acceptable salt, co-crystal, polymorph, hydrate, solvate, stereoisomer, or pro-drug of psilocybin. In some embodiments, the pharmaceutical composition comprises a delivery vehicle comprising the targeting peptide on an outer surface of the delivery vehicle. In some embodiments, the delivery vehicle comprises a hydrophilic surface and a hydrophobic volume, and the outer surface of the delivery vehicle comprises a hydrophilic outer surface. In some embodiments, the homing peptide is covalently linked to a saturated or unsaturated, substituted or unsubstituted, straight or branched carbon chain inserted into the hydrophobic volume of the delivery vehicle. In some embodiments, the hydrophobic volume of the delivery vehicle comprises psilocybin, or a pharmaceutically acceptable salt, co-crystal, polymorph, hydrate, solvate, stereoisomer, or pro-drug of psilocybin. In some embodiments, the delivery vehicle encloses psilocybin, or a pharmaceutically acceptable salt, co-crystal, polymorph, hydrate, solvate, stereoisomer, or pro-drug of psilocybin. In some embodiments, the delivery vehicle comprises a surfactant, a phospholipid, or a combination thereof. In some embodiments, the delivery vehicle comprises a micelle, a liposome, a bilayer sheet, or a combination thereof.

The molar ratio and/or the weight ratio of the targeting peptide and psilocybin, or a pharmaceutically acceptable salt, co-crystal, polymorph, hydrate, solvate, stereoisomer, or pro-drug of psilocybin, in the pharmaceutical composition can be about 10:1 to about 1:10. In some embodiments, the therapeutically effective amount of the pharmaceutical composition comprises about 1 mg to about 100 mg of psilocybin, or a pharmaceutically acceptable salt, co-crystal, polymorph, hydrate, solvate, stereoisomer, or pro-drug of psilocybin. In some embodiments, the therapeutically effective amount comprises about 1 mg to about 100 mg of the pharmaceutical composition. In some embodiments, the therapeutically effective amount of the pharmaceutical composition comprises about 10 μg to about 3000 μg of psilocybin, or a pharmaceutically acceptable salt, co-crystal, polymorph, hydrate, solvate, stereoisomer, or pro-drug of psilocybin, per kilogram of the body weight of the subject. In some embodiments, the therapeutically effective amount comprises about 10 μg to about 3000 μg of the pharmaceutical composition per kilogram of the body weight of the subject.

The method can, for example, comprise generating a desired effect in the subject in about 5 minutes to about 100 minutes. In some embodiments, the method comprises generating a desired effect in the subject in 25% to 75% of the time as compare to (1) when psilocybin, or a pharmaceutically acceptable salt, co-crystal, polymorph, hydrate, solvate, stereoisomer, or pro-drug of psilocybin, is administered in the subject in the absence of the targeting peptide, and/or (2) when psilocybin, or a pharmaceutically acceptable salt, co-crystal, polymorph, hydrate, solvate, stereoisomer, or pro-drug of psilocybin, is administered to the subject alone. In some embodiments, the desired effect lasts about 1 hour to about 12 hours in the subject. In some embodiments, the desired effect comprises a pain-relieving effect, a psychedelic, or a combination thereof.

In some embodiments, the maximum concentration (Cₘₐₓ) of psilocybin, or a pharmaceutically acceptable salt, co-crystal, polymorph, hydrate, solvate, stereoisomer, or pro-drug of psilocybin, in the blood of the subject is about 2 μg/ml to about 12 μg/ml. In some embodiments, the time to reach the maximum concentration (Tₘₐₓ) of psilocybin, or a pharmaceutically acceptable salt, co-crystal, polymorph, hydrate, solvate, stereoisomer, or pro-drug of psilocybin, in the blood of the subject is about 10 minutes to about 150 minutes. In some embodiments, the elimination half-life (T_{1/2}) of psilocybin, or a pharmaceutically acceptable salt, co-crystal, polymorph, hydrate, solvate, stereoisomer, or pro-drug of psilocybin, in the blood of the subject is about 20 minutes to about 200 minutes. In some embodiments, the Cₘₐₓ of psilocybin, or a pharmaceutically acceptable salt, co-crystal, polymorph, hydrate, solvate, stereoisomer, or pro-drug of psilocybin, in cell(s), tissue(s), organ(s), and/or environment(s) thereof of the target environment of the subject is about 2 μg/ml to about 12 μg/ml. In some embodiments, the Tₘₐₓ of psilocybin, or a pharmaceutically acceptable salt, co-crystal, polymorph, hydrate, solvate, stereoisomer, or pro-drug of psilocybin, in cell(s), tissue(s), organ(s), and/or environment(s) thereof of the target environment of the subject is about 10 minutes to about 150 minutes. In some embodiments, the elimination half-life (T_{1/2}) of psilocybin, or a pharmaceutically acceptable salt, co-crystal, polymorph, hydrate, solvate, stereoisomer, or pro-drug of psilocybin, in cell(s), tissue(s), organ(s), and/or environment(s) thereof of the target environment of the subject is about 20 minutes to about 200 minutes. In some embodiments, the Cₘₐₓ of psilocybin, or a pharmaceutically acceptable salt, co-crystal, polymorph, hydrate, solvate, stereoisomer, or pro-drug of psilocybin, in cell(s), tissue(s), organ(s), and/or environment(s) thereof of the target environment of the subject is about 50% to about 150% of the maximum concentration of psilocybin, or a pharmaceutically acceptable salt, co-crystal, polymorph, hydrate, solvate, stereoisomer, or pro-drug of psilocybin, in the blood of the subject. In some embodiments, the Tₘₐₓ of psilocybin, or a pharmaceutically acceptable salt, co-crystal, polymorph, hydrate, solvate, stereoisomer, or pro-drug of psilocybin, in cell(s), tissue(s), organ(s), and/or environment(s) thereof of the target environment of the subject is about 100% to about 200% of the time to reach the maximum concentration of psilocybin, or a pharmaceutically acceptable salt, co-crystal, polymorph, hydrate, solvate, stereoisomer, or pro-drug of psilocybin, in the blood of the subject. In some embodiments, the elimination half-life (T_{1/2}) of psilocybin, or a pharmaceutically acceptable salt, co-crystal, polymorph, hydrate, solvate, stereoisomer, or pro-drug of psilocybin, in cell(s), tissue(s), organ(s), and/or environment(s) thereof of the target environment of the subject is about 100% to about 200% of the elimination half-life of psilocybin, or a pharmaceutically acceptable salt, co-crystal, polymorph, hydrate, solvate, stereoisomer, or pro-drug of psilocybin, in the blood of the subject. In some embodiments, the Cₘₐₓ of psilocybin, or a pharmaceutically acceptable salt, co-crystal, polymorph, hydrate, solvate, stereoisomer, or pro-drug of psilocybin, in cell(s), tissue(s), organ(s), and/or environment(s) thereof of the target environment of the subject is about 50% to about 150% of the maximum concentration of psilocybin, or a pharmaceutically acceptable salt, co-crystal, polymorph, hydrate, solvate, stereoisomer, or pro-drug of psilocybin, in cell(s), tissue(s), organ(s), and/or environment(s) thereof of the target environment of the subject when psilocybin, or a pharmaceutically acceptable salt, co-crystal, polymorph, hydrate, solvate, stereoisomer, or pro-drug of psilocybin, is administered in the absence of the targeting peptide. In some embodiments, the Cₘₐₓ of psilocybin, or a pharmaceutically acceptable salt, co-crystal, polymorph, hydrate, solvate, stereoisomer, or pro-drug of psilocybin, in cell(s), tissue(s), organ(s), and/or environment(s) thereof of the target environment of the subject is about 50% to about 150% of the maximum concentration of psilocybin, or a pharmaceutically acceptable salt, co-crystal, polymorph, hydrate, solvate, stereoisomer, or pro-drug of psilocybin, in cell(s), tissue(s), organ(s), and/or environment(s) thereof of the target environment of the subject when psilocybin, or a pharmaceutically acceptable salt, co-crystal, polymorph, hydrate, solvate, stereoisomer, or pro-drug of psilocybin, is administered alone. In some embodiments, the Tₘₐₓ of psilocybin, or a pharmaceutically acceptable salt, co-crystal, polymorph, hydrate, solvate, stereoisomer, or pro-drug of psilocybin, in cell(s), tissue(s), organ(s), and/or environment(s) thereof of the target environment of the subject is about 100% to about 200% of the time to reach the maximum concentration of psilocybin, or a pharmaceutically acceptable salt, co-crystal, polymorph, hydrate, solvate, stereoisomer, or pro-drug of psilocybin, in cell(s), tissue(s), organ(s), and/or environment(s) thereof of the target environment of the subject (1) when psilocybin, or a pharmaceutically acceptable salt, co-crystal, polymorph, hydrate, solvate, stereoisomer, or pro-drug of psilocybin, is administered in the absence of the targeting peptide, and/or (2) when psilocybin, or a pharmaceutically acceptable salt, co-crystal, polymorph, hydrate, solvate, stereoisomer, or pro-drug of psilocybin, is administered alone.

In some embodiments, the elimination half-life (T_{1/2}) of psilocybin, or a pharmaceutically acceptable salt, co-crystal, polymorph, hydrate, solvate, stereoisomer, or pro-drug of psilocybin, in cell(s), tissue(s), organ(s), and/or environment(s) thereof of the target environment of the subject is about 100% to about 200% of the elimination half-life of psilocybin, or a pharmaceutically acceptable salt, co-crystal, polymorph, hydrate, solvate, stereoisomer, or pro-drug of psilocybin, in cell(s), tissue(s), organ(s), and/or environment(s) thereof of the target environment of the subject (1) when psilocybin, or a pharmaceutically acceptable salt, co-crystal, polymorph, hydrate, solvate, stereoisomer, or pro-drug of psilocybin, is administered alone, and/or (2) when psilocybin, or a pharmaceutically acceptable salt, co-crystal, polymorph, hydrate, solvate, stereoisomer, or pro-drug of psilocybin, is administered in the absence of the targeting peptide.

Cell(s), the tissue(s), and/or the organ of the target environment can comprise damaged and/or inflamed cell(s), tissue(s), or organ(s). In some embodiments, the cell(s), the tissue(s), and/or the organ(s) of the target environment comprises the brain, the white matter, the gray matter, the brainstem, the cerebellum, the diencephalon, the cerebrum, the spinal cord, the cranial nerve, cell(s) of any of the preceding, tissue(s) of any of the preceding, or a combination thereof.

In some embodiments, the administering comprises administering to the subject the therapeutically effective amount of the pharmaceutical composition orally, intravenously, or a combination thereof. In some embodiments, the method comprises administering to the subject one or more additional therapeutic agents, administering to the subject one or more cancer therapies, or both. The one or more additional therapeutic agents can, for example, comprise a radiotherapeutic agent, an anti-immunosuppressive agent or immunostimulatory agent, a chemotherapeutic agent, or a combination thereof. In some embodiments, the one or more additional therapeutic agents comprise an anti-PD-1 agent, an anti-PD-L1 agent, an anti-CTLA4 agent, an anti-TIM-3 agent, an anti-LAG-3 agent, a GITR (glucocorticoid-induced TNFR-related protein) stimulating agent, an anti-IDO agent, an anti-ICOS agent, an anti-OX40 agent, an anti-CSF1R agent, a chemokine signaling agent, a cytokine signal stimulating agent, or a combination thereof. In some embodiments, the one or more additional therapeutic agents comprise bevacizumab, pembrolizumab, nivolumab, PDR001, REGN2810 (SAR-439684), BGB-A317, BI 754091, IBI308, INCSHR-1210, JNJ-63723283, JS-001, MEDI0680 (AMP-514), MGA-012, PF-06801591, REGN-2810, TSR-042, atezolizumab, avelumab, CX-072, durvalumab, FAZ053, LY3300054, PD-L1 millamolecule, atezolizumab, durvalumab, avelumab, LY3300054, aminoglutethimide, amsacrine, anastrozole, asparaginase, beg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, vinorelbine, or a combination thereof. In some embodiments, the one or more cancer therapies comprise surgery, chemotherapy, radiotherapy, immunotherapy, or a combination thereof. In some embodiments, the one or more additional therapeutic agents and/or the one or more cancer therapies are administered to the subject concurrently with, before, or after, the administration of the pharmaceutical composition to the subject.

Disclosed herein include pharmaceutical compositions for use in the treatment of a proliferative disease and pharmaceutical compositions for use in alleviating one or more symptoms of a proliferative disease. The pharmaceutical compositions comprise, in some embodiments, a targeting peptide associated with psilocybin, or a pharmaceutically acceptable salt, co-crystal, polymorph, hydrate, solvate, stereoisomer, or pro-drug thereof, the targeting peptide comprises an amino acid sequence of any one of SEQ ID NOs: 1-22, and where the targeting peptide is capable of delivering the psilocybin, or the pharmaceutically acceptable salt, co-crystal, polymorph, hydrate, solvate, stereoisomer, or pro-drug of psilocybin, to a target environment of a subject.

The one or more symptoms can, for example, comprise pain. In some embodiments, the pain is bone pain, joint pain, back pain, neck pain, pain caused by spinal cord compression, or a combination thereof. In some embodiments, the pain is an acute pain, a chronic pain, or a combination thereof. In some embodiments, the pain is a somatic pain, a neuropathic pain, a visceral pain, or a combination thereof. In some embodiments, the proliferative disease is cancer, for example, carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, or a combination thereof.

The disease can be a solid tumor, optionally the solid tumor is neuroblastoma, Ewing sarcoma or Wilms tumor. In some embodiments, the disease is a liquid tumor. In some embodiments, the targeting peptide is a CNS targeting peptide. In some embodiments, the target environment is the nervous system, and optionally the nervous system is the CNS.

In some embodiments, the targeting peptide is directly associated with psilocybin, or a pharmaceutically acceptable salt, co-crystal, polymorph, hydrate, solvate, stereoisomer, or pro-drug of psilocybin. In some embodiments, the targeting peptide is covalently attached with psilocybin, or a pharmaceutically acceptable salt, co-crystal, polymorph, hydrate, solvate, stereoisomer, or pro-drug of psilocybin. In some embodiments, the targeting peptide is covalently attached with psilocybin, or a pharmaceutically acceptable salt, co-crystal, polymorph, hydrate, solvate, stereoisomer, or pro-drug of psilocybin, via a saturated or unsaturated, substituted or unsubstituted, straight or branched carbon chain. In some embodiments, the targeting peptide is conjugated to psilocybin, or a pharmaceutically acceptable salt, co-crystal, polymorph, hydrate, solvate, stereoisomer, or pro-drug of psilocybin. In some embodiments, the targeting peptide is non-covalently attached with psilocybin, or a pharmaceutically acceptable salt, co-crystal, polymorph, hydrate, solvate, stereoisomer, or pro-drug of psilocybin. In some embodiments, the targeting peptide is indirectly associated with psilocybin, or a pharmaceutically acceptable salt, co-crystal, polymorph, hydrate, solvate, stereoisomer, or pro-drug of psilocybin. In some embodiments, the pharmaceutical composition comprises a delivery vehicle comprising the targeting peptide on an outer surface of the delivery vehicle. In some embodiments, the delivery vehicle comprises a hydrophilic surface and a hydrophobic volume, and the outer surface of the delivery vehicle comprises a hydrophilic outer surface. In some embodiments, the homing peptide is covalently linked to a saturated or unsaturated, substituted or unsubstituted, straight or branched carbon chain inserted into the hydrophobic volume of the delivery vehicle. In some embodiments, the hydrophobic volume of the delivery vehicle comprises psilocybin, or a pharmaceutically acceptable salt, co-crystal, polymorph, hydrate, solvate, stereoisomer, or pro-drug of psilocybin. In some embodiments, the delivery vehicle encloses psilocybin, or a pharmaceutically acceptable salt, co-crystal, polymorph, hydrate, solvate, stereoisomer, or pro-drug of psilocybin. In some embodiments, the delivery vehicle comprises a surfactant, and/or a phospholipid. In some embodiments, the delivery vehicle comprises a micelle, a liposome, a bilayer sheet, or a combination thereof.

The molar ratio and/or the weight ratio of the targeting peptide and psilocybin, or a pharmaceutically acceptable salt, co-crystal, polymorph, hydrate, solvate, stereoisomer, or pro-drug of psilocybin, in the pharmaceutical composition can be about 10:1 to about 1:10. In some embodiments, the pharmaceutical composition comprises a therapeutically effective amount of about 1 mg to about 100 mg of psilocybin, or a pharmaceutically acceptable salt, co-crystal, polymorph, hydrate, solvate, stereoisomer, or pro-drug of psilocybin. In some embodiments, the pharmaceutical composition comprises a therapeutically effective amount of about 1 mg to about 100 mg of the pharmaceutical composition. In some embodiments, the pharmaceutical composition comprises a therapeutically effective amount of about 10 μg to 3000 μg of psilocybin, or a pharmaceutically acceptable salt, co-crystal, polymorph, hydrate, solvate, stereoisomer, or pro-drug of psilocybin, per kilogram of the body weight of a subject being administered the pharmaceutical composition. In some embodiments, the pharmaceutical composition comprises a therapeutically effective amount of about 10 μg to 3000 μg of the pharmaceutical composition per kilogram of the body weight of a subject being administered the pharmaceutical composition. In some embodiments, the therapeutically effective amount is capable of generating a desired effect in a subject being administered the pharmaceutical composition in about 5 minutes to about 100 minutes.

The therapeutically effective amount can be capable of generating a desired effect in a subject being administered the pharmaceutical composition in 25% to 75% of the time for generating the desired effect (1) when psilocybin, or a pharmaceutically acceptable salt, co-crystal, polymorph, hydrate, solvate, stereoisomer, or pro-drug of psilocybin, is administered in the subject in the absence of the targeting peptide and/or (2) when psilocybin, or a pharmaceutically acceptable salt, co-crystal, polymorph, hydrate, solvate, stereoisomer, or pro-drug of psilocybin, is administered to the subject alone. In some embodiments, the desired effect lasts about 1 hour to about 12 hours in the subject. In some embodiments, the desired effect comprises a pain-relieving effect, a psychedelic, or a combination thereof. In some embodiments, the Cₘₐₓ of psilocybin, or a pharmaceutically acceptable salt, co-crystal, polymorph, hydrate, solvate, stereoisomer, or pro-drug of psilocybin, in the blood of a subject being administered the pharmaceutical composition is about 2 μg/ml to about 12 μg/ml. In some embodiments, the Tₘₐₓ of psilocybin, or a pharmaceutically acceptable salt, co-crystal, polymorph, hydrate, solvate, stereoisomer, or pro-drug of psilocybin, in the blood of a subject being administered the pharmaceutical composition is about 10 minutes to about 150 minutes. In some embodiments, the elimination half-life (T_{1/2}) of psilocybin, or a pharmaceutically acceptable salt, co-crystal, polymorph, hydrate, solvate, stereoisomer, or pro-drug of psilocybin, in the blood of a subject being administered the pharmaceutical composition is about 20 minutes to about 200 minutes. In some embodiments, the Cₘₐₓ of psilocybin, or a pharmaceutically acceptable salt, co-crystal, polymorph, hydrate, solvate, stereoisomer, or pro-drug of psilocybin, in cell(s), tissue(s), organ(s), and/or environment(s) thereof of the target environment of a subject being administered the pharmaceutical composition is about 2 μg/ml to about 12 μg/ml. In some embodiments, the Tₘₐₓ of psilocybin, or a pharmaceutically acceptable salt, co-crystal, polymorph, hydrate, solvate, stereoisomer, or pro-drug of psilocybin, in cell(s), tissue(s), organ(s), and/or environment(s) thereof of the target environment of a subject being administered the pharmaceutical composition is about 10 minutes to about 150 minutes. In some embodiments, the elimination half-life (T_{1/2}) of psilocybin, or a pharmaceutically acceptable salt, co-crystal, polymorph, hydrate, solvate, stereoisomer, or pro-drug of psilocybin, in cell(s), tissue(s), organ(s), and/or environment(s) thereof of the target environment of a subject being administered the pharmaceutical composition is about 20 minutes to about 200 minutes. In some embodiments, the Cₘₐₓ of psilocybin, or a pharmaceutically acceptable salt, co-crystal, polymorph, hydrate, solvate, stereoisomer, or pro-drug of psilocybin, in cell(s), tissue(s), organ(s), and/or environment(s) thereof of the target environment of a subject being administered the pharmaceutical composition is about 50% to about 150% of the maximum concentration of psilocybin, or a pharmaceutically acceptable salt, co-crystal, polymorph, hydrate, solvate, stereoisomer, or pro-drug of psilocybin, in the blood of the subject. In some embodiments, the Tₘₐₓ of psilocybin, or a pharmaceutically acceptable salt, co-crystal, polymorph, hydrate, solvate, stereoisomer, or pro-drug of psilocybin, in cell(s), tissue(s), organ(s), and/or environment(s) thereof of the target environment of a subject being administered the pharmaceutical composition is about 100% to about 200% of the time to reach the maximum concentration of psilocybin, or a pharmaceutically acceptable salt, co-crystal, polymorph, hydrate, solvate, stereoisomer, or pro-drug of psilocybin, in the blood of the subject. In some embodiments, the elimination half-life (T_{1/2}) of psilocybin, or a pharmaceutically acceptable salt, co-crystal, polymorph, hydrate, solvate, stereoisomer, or pro-drug of psilocybin, in cell(s), tissue(s), organ(s), and/or environment(s) thereof of the target environment of a subject being administered the pharmaceutical composition is about 100% to about 200% of the elimination half-life of psilocybin, or a pharmaceutically acceptable salt, co-crystal, polymorph, hydrate, solvate, stereoisomer, or pro-drug of psilocybin, in the blood of the subject. In some embodiments, the Cₘₐₓ of psilocybin, or a pharmaceutically acceptable salt, co-crystal, polymorph, hydrate, solvate, stereoisomer, or pro-drug of psilocybin, in cell(s), tissue(s), organ(s), and/or environment(s) thereof of the target environment of a subject being administered the pharmaceutical composition is about 50% to about 150% of the maximum concentration of psilocybin in cell(s), tissue(s), organ(s), and/or environment(s) thereof of the target environment of the subject (1) when psilocybin, or a pharmaceutically acceptable salt, co-crystal, polymorph, hydrate, solvate, stereoisomer, or pro-drug of psilocybin, is administered in the absence of the targeting peptide, and/or (2) when psilocybin, or a pharmaceutically acceptable salt, co-crystal, polymorph, hydrate, solvate, stereoisomer, or pro-drug of psilocybin, is administered alone to the subject. In some embodiments, the Tₘₐₓ of psilocybin, or a pharmaceutically acceptable salt, co-crystal, polymorph, hydrate, solvate, stereoisomer, or pro-drug of psilocybin, in cell(s), tissue(s), organ(s), and/or environment(s) thereof of the target environment of a subject being administered the pharmaceutical composition is about 100% to about 200% of the time to reach the maximum concentration of psilocybin in cell(s), tissue(s), organ(s), and/or environment(s) thereof of the target environment of the subject (1) when psilocybin, or a pharmaceutically acceptable salt, co-crystal, polymorph, hydrate, solvate, stereoisomer, or pro-drug of psilocybin, is administered in the absence of the targeting peptide, and/or when the psilocybin, or a pharmaceutically acceptable salt, co-crystal, polymorph, hydrate, solvate, stereoisomer, or pro-drug of psilocybin, is administered alone. In some embodiments, the elimination half-life (T_{1/2}) of psilocybin in cell(s), tissue(s), organ(s), and/or environment(s) thereof of the target environment of a subject being administered the pharmaceutical composition is about 100% to about 200% of the elimination half-life of psilocybin in cell(s), tissue(s), organ(s), and/or environment(s) thereof of the target environment of the subject (1) when psilocybin, or a pharmaceutically acceptable salt, co-crystal, polymorph, hydrate, solvate, stereoisomer, or pro-drug of psilocybin, is administered alone, and/or (2) psilocybin, or a pharmaceutically acceptable salt, co-crystal, polymorph, hydrate, solvate, stereoisomer, or pro-drug of psilocybin, is administered in the absence of the targeting peptide.

Cell(s), tissue(s), and/or organ(s) of the target environment can comprise damaged and/or inflamed cell(s), tissue(s), and/or organ(s). In some embodiments, the cell(s), the tissue(s), and/or the organ(s) of the target environment comprises the brain, the white matter, the gray matter, the brainstem, the cerebellum, the diencephalon, the cerebrum, the spinal cord, the cranial nerve, cell(s) of any of the preceding, tissue(s) of any of the preceding, or a combination thereof. In some embodiments, the pharmaceutical composition is formulated for oral administration, intravenous administration, or a combination thereof. In some embodiments, the pharmaceutical composition comprises administering to the subject one or more additional therapeutic agents. In some embodiments, the one or more additional therapeutic agents comprise a radiotherapeutic agent, an anti-immunosuppressive agent or immunostimulatory agent, a chemotherapeutic agent, or a combination thereof. In some embodiments, the one or more additional therapeutic agents comprise an anti-PD-1 agent, an anti-PD-L1 agent, an anti-CTLA4 agent, an anti-TIM-3 agent, an anti-LAG-3 agent, a GITR (glucocorticoid-induced TNFR-related protein) stimulating agent, an anti-IDO agent, an anti-ICOS agent, an anti-OX40 agent, an anti-CSF1R agent, a chemokine signaling agent, a cytokine signal stimulating agent, or a combination thereof. In some embodiments, the one or more additional therapeutic agents comprise bevacizumab, pembrolizumab, nivolumab, PDR001, REGN2810 (SAR-439684), BGB-A317, BI 754091, IBI308, INCSHR-1210, JNJ-63723283, JS-001, MEDI0680 (AMP-514), MGA-012, PF-06801591, REGN-2810, TSR-042, atezolizumab, avelumab, CX-072, durvalumab, FAZ053, LY3300054, PD-L1 millamolecule, atezolizumab, durvalumab, avelumab, LY3300054, aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, vinorelbine, or a combination thereof.

Disclosed herein include kits for treating a proliferative disease, and/or preventing, alleviating or delaying the onset of one or more symptoms of a proliferative disease. In some embodiments, the kit comprises any pharmaceutical composition disclosed herein and instructions for one or more of (1) using the pharmaceutical composition to treat a proliferative disease, (2) alleviating one or more symptoms of a proliferative disease, (3) preventing the onset of one or more symptoms of a proliferative disease, and (4) delaying the onset of one or more symptoms of a proliferative disease.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the FIGURES, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include methods of treating a proliferative disease, such as cancer, in a subject in need thereof. In some embodiments, a method of treating the proliferative disease in a subject in need thereof comprises: administering to the subject a therapeutically effective amount of a pharmaceutical composition. The pharmaceutical composition can comprise a targeting peptide (e.g., a central nervous system (CNS) homing peptide) comprising an amino acid sequence of any one of SEQ ID NOs: 1-22 associated with psilocybin, or a pharmaceutically acceptable salt, co-crystal, polymorph, hydrate, solvate, stereoisomer, or pro-drug thereof. In some embodiments, the administering comprises administering to the subject the therapeutically effective amount of the pharmaceutical composition orally, intravenously, or a combination thereof.

Disclosed herein include embodiments of a pharmaceutical composition for the treating of a proliferative disease. For example, the pharmaceutical composition can comprise a targeting peptide (e.g., central nervous system (CNS) homing peptide) associated with psilocybin, or a pharmaceutically acceptable salt, co-crystal, polymorph, hydrate, solvate, stereoisomer, or pro-drug thereof. The targeting peptide can comprise an amino acid sequence of any one of SEQ ID NOs: 1-22. In some embodiments, the pharmaceutical composition comprises the targeting peptide associated with psilocybin, or a pharmaceutically acceptable salt, co-crystal, polymorph, hydrate, solvate, stereoisomer, or pro-drug thereof. The targeting peptide can comprise an amino acid sequence of any one of SEQ ID NOs: 1-22. In some embodiments, the pharmaceutical composition is formulated for oral administration, intravenous administration, or a combination thereof. Disclosed herein include embodiments of a kit comprising any pharmaceutical composition disclosed herein and instructions for using the pharmaceutical composition to treat a proliferative disease such as cancer.

### Definitions

As used herein, the term “administering” refers to oral administration, administration as a suppository, topical contact, intravenous, intraperitoneal, intramuscular, intralesional, intranasal or subcutaneous administration, or the implantation of a slow-release device e.g., a mini-osmotic pump, to a subject. Administration can be by any suitable route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery of pharmaceutical compositions and therapeutic substances disclosed herein include, but are not limited to, the use of liposomal formulations, intravenous infusion, transdermal patches, or a combination thereof.

As used herein, the term “daily dose” or “daily dosage” refers to a total amount of a pharmaceutical composition or a therapeutic agent that is to be taken within 24 hours.

As used herein, the term “delivery” refers to approaches, formulations, technologies, and systems for transporting a pharmaceutical composition or a therapeutic agent into the body of a patient as needed to safely achieve its desired therapeutic effect. In some embodiments, an effective amount of the composition or agent is formulated for delivery into the blood stream of a patient.

As used herein, the term “formulated” or “formulation” refers to the process in which different chemical substances, including one or more pharmaceutically active ingredients, are combined to produce a dosage form. In some embodiments, two or more pharmaceutically active ingredients can be co-formulated into a single dosage form or combined dosage unit, or formulated separately and subsequently combined into a combined dosage unit. A sustained release formulation is a formulation which is designed to slowly release a therapeutic agent in the body over an extended period of time, whereas an immediate release formulation is a formulation which is designed to quickly release a therapeutic agent in the body over a shortened period of time.

As used herein, the term “hydrate” refers to a complex formed by combination of water molecules with molecules or ions of the solute. As used herein, the term “solvate” refers to a complex formed by combination of solvent molecules with molecules or ions of the solute. The solvent can be an organic compound, an inorganic compound, or a mixture of both. Solvate is meant to include hydrate, hemi-hydrate, channel hydrate etc. Some examples of solvents include, but are not limited to, methanol, N,N-dimethylformamide, tetrahydrofuran, dimethylsulfoxide, and water.

As used herein, the term “patient” refers to a subject having a disease. In some embodiments, the patient is a human or an animal. In some embodiments, the patient is a mammal.

As used herein, the term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile.

As used herein, the term “pharmaceutically acceptable carrier” refers to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to a diseased tissue or a tissue adjacent to the diseased tissue. Carriers or excipients can be used to produce compositions. The carriers or excipients can be chosen to facilitate administration of a drug or pro-drug. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

As used herein, the term “pharmaceutically acceptable salt” refers to any acid or base addition salt whose counter-ions are non-toxic to the patient in pharmaceutical doses of the salts. A host of pharmaceutically acceptable salts are well known in the pharmaceutical field. If pharmaceutically acceptable salts of the compounds of this disclosure are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate, undecanoate, hydrohalides (e.g., hydrochlorides and hydrobromides), sulphates, phosphates, nitrates, sulphamates, malonates, salicylates, methylene-bis-b-hydroxynaphthoates, gentisates, isethionates, di-p-toluoyltartrates, ethanesulphonates, cyclohexylsulphamates, quinates, and the like. Pharmaceutically acceptable base addition salts include, without limitation, those derived from alkali or alkaline earth metal bases or conventional organic bases, such as triethylamine, pyridine, piperidine, morpholine, N-methylmorpholine, ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

The terms “prevent,” “preventing,” “prevention,” and grammatical variations thereof as used herein, refer to a method of partially or completely delaying or precluding the onset or recurrence of a disorder or condition and/or one or more of its attendant symptoms or barring a subject from acquiring or reacquiring a disorder or condition or reducing a subject's risk of acquiring or requiring a disorder or condition or one or more of its attendant symptoms.

As used herein, the term “therapeutically effective” or “effective amount” indicates that a compound or material or amount of the compound or material when administered is sufficient or effective to prevent, alleviate, or ameliorate one or more symptoms of a disease, disorder or medical condition being treated, and/or to prolong the survival of the subject being treated. The therapeutically effective amount will vary depending on the compound, the disease, disorder or condition and its severity and the age, weight, etc., of the mammal to be treated. The dosage can be conveniently administered, e.g., in divided doses up to four times a day or in sustained-release form.

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments as described herein may be applied preventively, prophylactically, palliatively or remedially.

### Targeted Delivery of Psilocybin and Analogues Thereof

Disclosed herein include methods for treating a proliferative disease in a patient or subject in need thereof. In some embodiments, the method comprises administering to the subject a therapeutically effective amount of a pharmaceutical composition (or a pharmaceutically acceptable carrier, pharmaceutically acceptable salt, co-crystal, polymorph, hydrate, or solvate thereof). The pharmaceutical composition can comprise a targeting peptide, for example a central nervous system (CNS) homing or targeting peptide. The targeting peptide can comprise, or consist of, one of the amino acid sequences provided in Table 1. In some embodiments, the targeting peptide comprises, or consists of, an amino acid sequence having one, two, three, four, or five amino acid mismatches as compared to one of the amino acid sequences provided in Table 1. In some embodiments, the targeting peptide comprises, or consists of, an amino acid sequence having a deletion of one, two, three, four, or five amino acids as compared to one of the amino acid sequences provided in Table 1. In some embodiments, the targeting peptide comprises, or consists of, an amino acid sequence having one, two, three, four, or five additional amino acids (e.g., additions to the N-terminal, internal additions, and additions to the C-terminal) as compared to one of the amino acid sequences provided in Table 1. CNS homing or targeting peptides have been described in US2019/0359651, the content of which is incorporated herein by reference in its entirety. The pharmaceutical composition can comprise psilocybin or an analogue thereof (or a pharmaceutically acceptable carrier, pharmaceutically acceptable salt, co-crystal, polymorph, hydrate, solvate, stereoisomer, or pro-drug of psilocybin or an analogue thereof). The targeting peptide can be associated with psilocybin, or an analogue thereof. In some embodiments, the administering comprises administering to the subject the therapeutically effective amount of the pharmaceutical composition orally, intravenously, or a combination thereof.

TABLE 1
CNS homing or targeting peptide sequences

| Amino Acid Sequence | SED ID NO |
|---|---|
| KRSS | 1 |
| PGESS | 2 |
| SLTQ | 3 |
| AMGN | 4 |
| GDRLV | 5 |
| RGGKRSS | 6 |
| SLTQDQG | 7 |
| ADAQADV | 8 |
| RAKGRDA | 9 |
| KASRLGR | 10 |
| RGASMDG | 11 |
| GRPGESS | 12 |
| SRTEGDV | 13 |
| CRGGKRSSC | 14 |
| CSLTQDQGC | 15 |
| CADAQADVC | 16 |
| CRAKGRDAC | 17 |
| CKASRLGRC | 18 |
| CRGASMDGC | 19 |
| CGRPGESSC | 20 |
| CSRTEGDVC | 21 |
| CSGRRGKSC | 22 |

### Pharmaceutical Compositions

A pharmaceutical composition (or a pharmaceutically acceptable carrier, pharmaceutically acceptable salt, co-crystal, polymorph, hydrate, or solvate thereof) of the present disclosure can be for use in treating a proliferative disease. The pharmaceutical composition can be formulated, for example, for oral administration, intravenous administration, or a combination thereof. A kit can comprise any pharmaceutical composition disclosed herein and instructions for using the pharmaceutical composition to treat a proliferative disease.

Psilocybin and Targeting Peptide

In some embodiments, a targeting peptide, for example a CNS homing peptide, is directly associated with psilocybin or an analogue thereof (or a pharmaceutically acceptable salt, co-crystal, polymorph, hydrate, solvate, stereoisomer, or pro-drug of psilocybin or an analogue thereof). For example, a targeting peptide can be covalently attached with psilocybin. The targeting peptide can be conjugated to psilocybin. The targeting peptide can be covalently attached with psilocybin via a saturated or unsaturated, substituted or unsubstituted, straight or branched carbon chain. The targeting peptide can be non-covalently attached with psilocybin. In some embodiments, the targeting peptide is indirectly associated with psilocybin. For example, the pharmaceutical composition can comprise a delivery vehicle comprising the targeting peptide associated with psilocybin.

In some embodiments, the pharmaceutical composition, or a pharmaceutically acceptable carrier, pharmaceutically acceptable salt, co-crystal, polymorph, hydrate, or solvate thereof, can comprise two or more targeting peptides. Two targeting peptides can be associated with psilocybin. One or more targeting peptide may not be associated with psilocybin. The pharmaceutical composition can comprise psilocybin and one or more analogous of psilocybin. In some embodiments, the pharmaceutical composition comprises two analogous of psilocybin. In some embodiments, psilocybin and/or analogous of psilocybin in the pharmaceutical composition can be associated with different targeting peptides. In some embodiments, psilocybin and/or one or more analogous of psilocybin in the pharmaceutical composition can be associated with an identical targeting peptide. For example, psilocybin and/or one or more analogous of psilocybin in the pharmaceutical composition is associated with different molecules of a targeting peptide. For example, a molecule of psilocybin and a molecule of an analogue of psilocybin in the pharmaceutical composition are associated with a molecule of the targeting peptide.

The pharmaceutical composition can comprise different numbers of the targeting peptide (e.g., CNS homing peptides) in different embodiments. For example, the pharmaceutical composition can comprise, comprise about, comprise at least, comprise at least about, comprise at most, or comprise at most about, 1, 2, 3, 4, 5, 6, 7, 8, 9, 10, 11, 12, 13, 14, 15, 16, 17, 18, 19, 20, 30, 40, 50, 60, 70, 80, 90, 100, or a number or a range between any two of these values, the targeting peptides. Two of the targeting peptides (and associated psilocybin and/or one or more analogues of psilocybin and/or one or more analogues of psilocybin) can be co-administered simultaneously or sequentially. Two, or more, targeting peptides in the pharmaceutical composition can be associated with psilocybin. All CNS homing peptides in the pharmaceutical composition can be associated with psilocybin. Two, or more, targeting peptides in the pharmaceutical composition can be associated with different psilocybin and/or one or more analogues of psilocybin. All targeting peptides in the pharmaceutical composition can be associated with different psilocybin and/or one or more analogues of psilocybin.

The pharmaceutical composition can comprise different numbers of psilocybin and/or one or more analogues of psilocybin in different embodiments. In some embodiments, the pharmaceutical composition comprises, comprises about, comprises at least, comprises at least about, comprises at most, or comprises at most about, 1, 2, 3, 4, 5, 6, 7, 8, 9, 10, 11, 12, 13, 14, 15, 16, 17, 18, 19, 20, 30, 40, 50, 60, 70, 80, 90, 100, or a number or a range between any two of these values, psilocybin and/or one or more analogues of psilocybin. Two, or more, psilocybin and/or one or more analogues of psilocybin can be co-administered simultaneously or sequentially. Two, or more, of psilocybin and/or one or more analogues of psilocybin in the pharmaceutical composition can be associated with molecules of a targeting peptide. All psilocybin and/or one or more analogues of psilocybin in the pharmaceutical composition can be associated with molecules of a targeting peptide. Two, or more, psilocybin and/or one or more analogues of psilocybin in the pharmaceutical composition can be associated with different targeting peptides. All psilocybin and/or one or more analogues of psilocybin in the pharmaceutical composition can be associated with different targeting peptides.

An analogue of psilocybin can have the structure of Formula I:

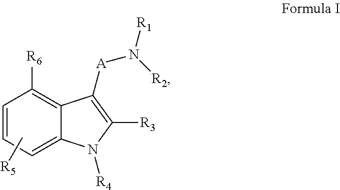

wherein:

A is C₁-C₄ alkylene, C₂-C₄ alkenylene, or C₂-C₄ alkynylene;

R₁ and R₂ are, independently for each occurrence, H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen (e.g., F, Cl, Br, I, or At), cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH;

R₃ is H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₃ is selected from the group consisting of halogen, C₁-C₈ alkylsulfonyl, formyl, COOH, hydroxy, C₁-C₈ alkoxyl, —SH, thio(C₂-C₈)alkyl, amino, C₁-C₈ alkylamino, and di(C₁-C₈ alkyl)amino;

R₄ is H, C₁-C₈ alkyl, C₂-C₈ alkenyl, or C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₄ is selected from the group consisting of C₁-C₈ alkylsulfonyl, formyl, hydroxy, C₁-C₈ alkoxyl, —SH, thio(C₂-C₈)alkyl, amino, C₁-C₈ alkylamino, and di(C₁-C₈ alkyl)amino;

R₅ represents 1-3 substituents, each of which is independently selected from the group consisting of C₁-C₈ alkyl, C₂-C₈ alkenyl, and C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, and di(C₁-C₈ alkyl)amino; or R₅ represents 1-3 substituents, each of which is independently selected from the group consisting of halogen, C₁-C₈ alkylsulfonyl, formyl, COOH, hydroxy, C₁-C₈ alkoxyl, —SH, thio(C₂-C₈)alkyl, amino, C₁-C₈ alkylamino, and di(C₁-C₈ alkyl)amino;

R₆ is phosphate, halogen, R₇ or OR₇; and

R₇ is C₁-C₈ alkyl, optionally substituted with 1-3 substituents, each of which is independently selected from the group consisting of halogen, cyano, hydroxy, oxo, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, COOH, and phosphate; or a pharmaceutically acceptable salt thereof. Analogues of psilocybin have been described in U.S. Patent Application Publication No. 2009/0318527, the content of which is incorporated herein by reference in its entirety.

Weight and Weight Ratio

The weight of the pharmaceutical composition can be different in different embodiments. In some embodiments, the weight of the pharmaceutical composition is, is about, is at least, is at least about, is at most, or is at most about, 10 μg, 20 μg, 30 μg, 40 μg, 50 μg, 60 g, 70 μg, 80 μg, 90 μg, 100 μg, 150 μg, 200 μg, 250 μg, 300 μg, 350 μg, 400 μg, 450 μg, 500 ag, 550 μg, 600 μg, 650 μg, 700 μg, 750 μg, 800 μg, 850 μg, 900 μg, 950 μg, 1000 μg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 550 mg, 600 mg, 650 mg, 700 mg, 750 mg, 800 mg, 850 mg, 900 mg, 950 mg, 1000 mg, 1 g, 2 g, 3 g, 4 g, 5 g, 6 g, 7 g, 8 g, 9 g, 10 g, or a number or a range between any two of these values. For example, the weight of the pharmaceutical composition can be about 1 mg to 100 mg.

The weight of psilocybin (or an analogue of psilocybin) in the pharmaceutical composition disclosed herein (or the total weight of two or more of psilocybin and/or one or more analogues of psilocybin, such as all psilocybin and/or one or more analogues of psilocybin) can be different in different embodiments. In some embodiments, the weight of psilocybin (or an analogue of psilocybin) in the pharmaceutical composition (or the total weight of two or more of psilocybin and/or one or more analogues of psilocybin, such as all psilocybin and/or one or more analogues of psilocybin in the pharmaceutical composition) is, is about, is at least, is at least about, is at most, or is at most about, 10 μg, 20 μg, 30 μg, 40 μg, 50 μg, 60 μg, 70 μg, 80 μg, 90 μg, 100 μg, 150 μg, 200 μg, 250 μg, 300 μg, 350 μg, 400 μg, 450 μg, 500 μg, 550 g, 600 g, 650 g, 700 g, 750 g, 800 g, 850 g, 900 g, 950 g, 1000 g, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 550 mg, 600 mg, 650 mg, 700 mg, 750 mg, 800 mg, 850 mg, 900 mg, 950 mg, 1000 mg, 1 g, 2 g, 3 g, 4 g, 5 g, 6 g, 7 g, 8 g, 9 g, 10 g, or a number or a range between any two of these values. For example, the pharmaceutical composition can comprise about 1 mg to about 100 mg of psilocybin.

The molar ratio of a targeting peptide (e.g., a CNS homing/targeting peptide) and psilocybin (or an analogue of psilocybin) in the pharmaceutical compositions disclosed herein (or the molar ratio of a targeting peptide and two or more of psilocybin and/or one or more analogues of psilocybin, such as all psilocybin and/or one or more analogues of psilocybin, in the pharmaceutical composition) can be different in different embodiments. In some embodiments, the molar ratio of a targeting peptide and psilocybin (or an analogue of psilocybin) in the pharmaceutical compositions (or the molar ratio of a targeting peptide and two or more of psilocybin and/or one or more analogues of psilocybin, such as all psilocybin and/or one or more analogues of psilocybin, in the pharmaceutical composition) is, is about, is at least, is at least about, is at most, or is at most about, 1:100, 1:99, 1:98, 1:97, 1:96, 1:95, 1:94, 1:93, 1:92, 1:91, 1:90, 1:89, 1:88, 1:87, 1:86, 1:85, 1:84, 1:83, 1:82, 1:81, 1:80, 1:79, 1:78, 1:77, 1:76, 1:75, 1:74, 1:73, 1:72, 1:71, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of these values. For example, the molar ratio of the targeting peptide and psilocybin in the pharmaceutical composition can be about 1:10 to about 10:1.

The weight ratio of a targeting peptide and psilocybin (or an analogue of psilocybin) in the pharmaceutical compositions disclosed herein (or the weight ratio of a targeting peptide and two or more of psilocybin and/or one or more analogues of psilocybin, such as all psilocybin and/or one or more analogues of psilocybin, in the pharmaceutical composition) can be different in different embodiments. In some embodiments, the weight ratio of a targeting peptide and psilocybin (or an analogue of psilocybin) in the pharmaceutical compositions (or the weight ratio of a targeting peptide and two or more of psilocybin and/or one or more analogues of psilocybin, such as all psilocybin and/or one or more analogues of psilocybin, in the pharmaceutical composition) is, is about, is at least, is at least about, is at most, or is at most about, 1:100, 1:99, 1:98, 1:97, 1:96, 1:95, 1:94, 1:93, 1:92, 1:91, 1:90, 1:89, 1:88, 1:87, 1:86, 1:85, 1:84, 1:83, 1:82, 1:81, 1:80, 1:79, 1:78, 1:77, 1:76, 1:75, 1:74, 1:73, 1:72, 1:71, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of these values. For example, the weight ratio of the targeting peptide and psilocybin in the pharmaceutical composition can be about 1:10 to about 10:1.

The molar ratio of psilocybin and an analogue of psilocybin (or the molar ratio of two analogues of psilocybin, or the molar ratio of one of psilocybin and/or one or more analogous of psilocybin and the remaining of psilocybin and/or one or more analogues of psilocybin)) in the pharmaceutical compositions disclosed herein can be different in different embodiments. In some embodiments, the molar ratio of one psilocybin and an analogue of psilocybin (or the molar ratio of two analogues of psilocybin, or the molar ratio of one of psilocybin and/or one or more analogous of psilocybin and the remaining of psilocybin and/or one or more analogues of psilocybin) in the pharmaceutical compositions is, is about, is at least, is at least about, is at most, or is at most about, 1:100, 1:99, 1:98, 1:97, 1:96, 1:95, 1:94, 1:93, 1:92, 1:91, 1:90, 1:89, 1:88, 1:87, 1:86, 1:85, 1:84, 1:83, 1:82, 1:81, 1:80, 1:79, 1:78, 1:77, 1:76, 1:75, 1:74, 1:73, 1:72, 1:71, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of these values. For example, the molar ratio of two psilocybin and an analogue of psilocybin in the pharmaceutical composition can be about 1:10 to about 10:1.

The weight ratio of one psilocybin and an analogue of psilocybin (or the molar ratio of two analogues of psilocybin, or the molar ratio of one of psilocybin and/or one or more analogous of psilocybin and the remaining of psilocybin and/or one or more analogues of psilocybin) in the pharmaceutical compositions disclosed herein can be different in different embodiments. In some embodiments, the weight ratio of one psilocybin and an analogue of psilocybin (or the molar ratio of two analogues of psilocybin, or the molar ratio of one of psilocybin and/or one or more analogous of psilocybin and the remaining of psilocybin and/or one or more analogues of psilocybin) in the pharmaceutical compositions is, is about, is at least, is at least about, is at most, or is at most about, 1:100, 1:99, 1:98, 1:97, 1:96, 1:95, 1:94, 1:93, 1:92, 1:91, 1:90, 1:89, 1:88, 1:87, 1:86, 1:85, 1:84, 1:83, 1:82, 1:81, 1:80, 1:79, 1:78, 1:77, 1:76, 1:75, 1:74, 1:73, 1:72, 1:71, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of these values. For example, the weight ratio of two of psilocybin and/or one or more analogues of psilocybin in the pharmaceutical composition can be about 1:10 to about 10:1.

Delivery Vehicles

The pharmaceutical compositions disclosed herein can comprise a delivery vehicle comprising the targeting peptide (for example a CNS homing peptide) on an outer surface of the delivery vehicle. The delivery vehicle can comprise a hydrophilic surface and a hydrophobic volume. The outer surface of the delivery vehicle can comprise a hydrophilic surface. In some embodiments, the targeting peptide is covalently linked to a saturated or unsaturated, substituted or unsubstituted, straight or branched carbon chain inserted into the hydrophobic volume of the delivery vehicle. The hydrophobic volume of the delivery vehicle can comprise psilocybin (or an analogue of psilocybin). The delivery vehicle can enclose or encircle psilocybin (or an analogue of psilocybin). In some embodiments, the delivery vehicle comprises a surfactant. The delivery vehicle can comprise a phospholipid. The delivery vehicle comprises a micelle, a liposome, a bilayer sheet, or a combination thereof.

Carbon Chain

The length of a carbon chain (such as the carbon chain covalently attached to the CNS homing peptide and psilocybin, or the carbon chain covalently attached to the CNS homing peptide and inserted into the hydrophobic volume of a delivery vehicle) can be different in different embodiments. In some embodiments, the length of a carbon chain is, is about, is at least, is at least about, is at most, or is at most about, 10 Å, 11 Å, 12 Å, 13 Å, 14 Å, 15 Å, 16 Å, 17 Å, 18 Å, 19 Å, 20 Å, 21 Å, 22 Å, 23 Å, 24 Å, 25 Å, 26 Å, 27 Å, 28 Å, 29 Å, 30 Å, 31 Å, 32 Å, 33 Å, 34 Å, 35 Å, 36 Å, 37 Å, 38 Å, 39 Å, 40 Å, 41 Å, 42 Å, 43 Å, 44 Å, 45 Å, 46 Å, 47 Å, 48 Å, 49 Å, 50 Å, 51 Å, 52 Å, 53 Å, 54 Å, 55 Å, 56 Å, 57 Å, 58 Å, 59 Å, 60 Å, 61 Å, 62 Å, 63 Å, 64 Å, 65 Å, 66 Å, 67 Å, 68 Å, 69 Å, 70 Å, 71 Å, 72 Å, 73 Å, 74 Å, 75 Å, 76 Å, 77 Å, 78 Å, 79 Å, 80 Å, 81 Å, 82 Å, 83 Å, 84 Å, 85 Å, 86 Å, 87 Å, 88 Å, 89 Å, 90 Å, 91 Å, 92 Å, 93 Å, 94 Å, 95 Å, 96 Å, 97 Å, 98 Å, 99 Å, 100 Å, 200 Å, 300 Å, 400 Å, 500 Å, 600 Å, 700 Å, 800 Å, 900 Å, 1000 Å, or a number or a range between any two of these values.

The carbon chain can comprise one or more carbon atoms, zero, one, or more oxygen atoms, zero, one or more nitrogen atoms, zero, one, or more sulfur atoms, or a combination thereof. In some embodiments, the carbon chain comprises, comprises about, comprises at least, comprises at least about, comprises at most, or comprises at most about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 2000, 3000, 4000, 5000, 6000, 7000, 8000, 9000, 10000, or a number or a range between any two of these values, carbon atom(s), oxygen atom(s), nitrogen atom(s), sulfur atom(s), or a combination thereof. The carbon chain can comprise one or more carbon atoms, zero, one, or more oxygen atoms, zero, one or more nitrogen atoms, zero, one, or more sulfur atoms, or a combination thereof, in the main chain of the carbon chain. In some embodiments, the carbon chain comprises, comprises about, comprises at least, comprises at least about, comprises at most, or comprises at most about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 2000, 3000, 4000, 5000, 6000, 7000, 8000, 9000, 10000, or a number or a range between any two of these values, carbon atom(s), oxygen atom(s), nitrogen atom(s), sulfur atom(s), or a combination thereof, in the main chain of the carbon chain.

### Therapeutic Efficacy and Pharmacokinetics

Therapeutically Effective Amount

The therapeutically effective amount of the pharmaceutical composition (or a component of the pharmaceutical composition, such as psilocybin) can be the amount of the pharmaceutical composition (or a component of the pharmaceutical composition) administered per time period. For example, the therapeutically effective amount of the pharmaceutical composition can be the daily dose or dosage. The therapeutically effective amount of the pharmaceutical composition can be administered all in one time (once per time period) or in several times, such as two times, three times, four times, five times or more throughout the time period In some embodiments, the time period is, is about, is at least, is at least about, is at most, or is at most about, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 2 weeks, 3 weeks, 4 weeks, 5 weeks, 1 month, 2 months, 3 months, 4 months, 5 months, 6 months, 7 months, 8 months, 9 months, 10 months, 11 months, 12 months, or a number or a range between any two of these values.

The therapeutically effective amount of the pharmaceutical composition can be different in different embodiments. In some embodiments, the therapeutically effective amount of the pharmaceutical composition is, is about, is at least, is at least about, is at most, or is at most about, 10 μg, 20 μg, 30 μg, 40 μg, 50 μg, 60 μg, 70 μg, 80 μg, 90 μg, 100 μg, 150 μg, 200 g, 250 g, 300 g, 350 g, 400 g, 450 g, 500 g, 550 g, 600 g, 650 g, 700 g, 750 ag, 800 g, 850 g, 900 g, 950 g, 1000 g, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 550 mg, 600 mg, 650 mg, 700 mg, 750 mg, 800 mg, 850 mg, 900 mg, 950 mg, 1000 mg, 1 g, 2 g, 3 g, 4 g, 5 g, 6 g, 7 g, 8 g, 9 g, 10 g, or a number or a range between any two of these values. For example, the therapeutically effective amount of the pharmaceutical composition can be about 1 mg to about 100 mg of the pharmaceutical composition.

The therapeutically effective amount of the pharmaceutical composition can comprise different amounts of psilocybin or an analogue or psilocybin (or different total amounts of two or more of psilocybin and/or one or more analogues of psilocybin, such as all psilocybin and/or one or more analogues of psilocybin) in the pharmaceutical composition in different embodiments. In some embodiments, the weight of psilocybin or an analogue of psilocybin (or the total amount of two or more of psilocybin and/or one or more analogues of psilocybin, or all psilocybin and/or one or more analogues of psilocybin) in the therapeutically effective amount of the pharmaceutical composition is, is about, is at least, is at least about, is at most, or is at most about, 10 μg, 20 μg, 30 μg, 40 μg, 50 μg, 60 μg, 70 μg, 80 μg, 90 μg, 100 μg, 150 g, 200 g, 250 g, 300 g, 350 g, 400 g, 450 g, 500 g, 550 g, 600 g, 650 g, 700 ag, 750 g, 800 g, 850 g, 900 g, 950 g, 1000 g, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 550 mg, 600 mg, 650 mg, 700 mg, 750 mg, 800 mg, 850 mg, 900 mg, 950 mg, 1000 mg, 1 g, 2 g, 3 g, 4 g, 5 g, 6 g, 7 g, 8 g, 9 g, 10 g, or a number or a range between any two of these values. For example, the therapeutically effective amount of the pharmaceutical composition can comprise about 1 mg to about 100 mg of psilocybin.

The therapeutically effective amount of the pharmaceutical composition can be different in different embodiments. For example, the therapeutically effective amount comprises about 10 μg to 3000 μg of the pharmaceutical composition per kilogram of the body weight of the subject (μg/kg) being administered the pharmaceutical composition. In some embodiments, the therapeutically effective amount comprises, comprises about, comprises at least, comprises at least about, comprises at most, or comprises at most about, 100 ng/kg, 200 ng/kg, 300 ng/kg, 400 ng/kg, 500 ng/kg, 600 ng/kg, 700 ng/kg, 800 ng/kg, 900 ng/kg, 1000 ng/kg, 2 μg/kg, 3 μg/kg, 4 μg/kg, 5 μg/kg, 6 μg/kg, 7 μg/kg, 8 μg/kg, 9 μg/kg, 10 μg/kg, 20 μg/kg, 30 μg/kg, 40 μg/kg, 50 μg/kg, 60 μg/kg, 70 μg/kg, 80 μg/kg, 90 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 1000 μg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5 mg/kg, 6 mg/kg, 7 mg/kg, 8 mg/kg, 9 mg/kg, 10 mg/kg, or a number or a range between any two of these values, of the pharmaceutical composition.

The therapeutically effective amount of the pharmaceutical composition can comprise different amounts of psilocybin or an analogue of psilocybin (or different total amounts of two or more of psilocybin and/or one or more analogues of psilocybin, such as all psilocybin and/or one or more analogues of psilocybin) in the pharmaceutical composition in different embodiments. For example, the therapeutically effective amount of the pharmaceutical composition can comprise about 10 μg to 3000 μg of psilocybin per kilogram of the body weight of the subject (g/kg) being administered the pharmaceutical composition. In some embodiments, the therapeutically effective amount of the pharmaceutical composition comprises, comprises about, comprises at least, comprises at least about, comprises at most, or comprises at most about, 100 ng/kg, 200 ng/kg, 300 ng/kg, 400 ng/kg, 500 ng/kg, 600 ng/kg, 700 ng/kg, 800 ng/kg, 900 ng/kg, 1000 ng/kg, 2 μg/kg, 3 μg/kg, 4 μg/kg, 5 μg/kg, 6 μg/kg, 7 g/kg, 8 μg/kg, 9 μg/kg, 10 μg/kg, 20 μg/kg, 30 μg/kg, 40 μg/kg, 50 μg/kg, 60 μg/kg, 70 μg/kg, 80 μg/kg, 90 μg/kg, 100 μg/kg, 150 μg/kg, 200 μg/kg, 250 μg/kg, 300 μg/kg, 350 μg/kg, 400 g/kg, 450 μg/kg, 500 μg/kg, 550 μg/kg, 600 μg/kg, 650 μg/kg, 700 μg/kg, 750 μg/kg, 800 g/kg, 850 μg/kg, 900 μg/kg, 950 μg/kg, 1000 μg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5 mg/kg, 6 mg/kg, 7 mg/kg, 8 mg/kg, 9 mg/kg, 10 mg/kg, or a number or a range between any two of these values, of psilocybin or an analogue of psilocybin (or different total amounts of two or more of psilocybin and/or one or more analogues of psilocybin, such as all psilocybin and/or one or more analogues of psilocybin) in the pharmaceutical composition.

Targeted delivery using a targeting peptide (e.g., a CNS homing peptide) can lower the amount of psilocybin (or an analogue of psilocybin) administered while achieving a desired concentration in cell(s), tissue(s), and/or organ(s) of the target environment affected by the disease, thus reducing undesired side effects which may arise from higher dosage levels. In some embodiments, the amount of psilocybin (or an analogue of psilocybin) administered with targeted delivery using a targeting peptide is lowered by, by about, by at least, by at least about, by at most, or by at most about, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or a number or a range between any two of these values.

The amount of psilocybin (or an analogue of psilocybin) in the therapeutically effective amount of the pharmaceutical composition can be less than (or the same as, or more than) a therapeutically effective amount of psilocybin (or an analogue of psilocybin) when psilocybin (or an analogue of psilocybin) is administered in the absence of the targeting peptide or when psilocybin (or an analogue of psilocybin) is administered alone. In some embodiments, the amount of psilocybin (or an analogue of psilocybin) in the therapeutically effective amount of the pharmaceutical composition is, is about, is at least, is at least about, is at most, or is at most about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 105%, 110%, 115%, 120%, 125%, 130%, 135%, 140%, 145%, 150%, 155%, 160%, 165%, 170%, 175%, 180%, 185%, 190%, 195%, 200%, or a number or a range between any two of these values, of a therapeutically effective amount of psilocybin (or an analogue of psilocybin) when psilocybin (or an analogue of psilocybin) is administered in the absence of the targeting peptide or when psilocybin (or an analogue of psilocybin) is administered alone. For example, the amount of psilocybin (or an analogue of psilocybin) in the therapeutically effective amount of the pharmaceutical composition is about 50% to about 150% of a therapeutically effective amount of psilocybin (or an analogue of psilocybin) when psilocybin (or an analogue of psilocybin) is administered in the absence of the targeting peptide. For example, the amount of psilocybin (or an analogue of psilocybin) in the therapeutically effective amount of the pharmaceutical composition is about 50% to about 150% of a therapeutically effective amount of psilocybin (or an analogue of psilocybin) when psilocybin (or an analogue of psilocybin) is administered alone.

Effects

The therapeutically effective amount of the pharmaceutical composition can treat a proliferative disease in the subject. The therapeutically effective amount of the pharmaceutical composition can generate (or result in) a desired effect in the subject. For example, the desired effect comprises a pain-relieving effect, a psychedelic, or a combination thereof. The therapeutically effective amount of the pharmaceutical composition can generate (or result in) a desired effect in the subject in different amounts of time after the therapeutically effective amount of the pharmaceutical composition in different embodiments. In some embodiments, the therapeutically effective amount of the pharmaceutical composition generates (or results) in a desired effect in the subject in, in about, in at least, in at least about, in at most, or in at most about, 1 min, 2 mins, 3 mins, 4 mins, 5 mins, 6 mins, 7 mins, 8 mins, 9 mins, 10 mins, 11 mins, 12 mins, 13 mins, 14 mins, 15 mins, 16 mins, 17 mins, 18 mins, 19 mins, 20 mins, 21 mins, 22 mins, 23 mins, 24 mins, 25 mins, 26 mins, 27 mins, 28 mins, 29 mins, 30 mins, 31 mins, 32 mins, 33 mins, 34 mins, 35 mins, 36 mins, 37 mins, 38 mins, 39 mins, 40 mins, 41 mins, 42 mins, 43 mins, 44 mins, 45 mins, 46 mins, 47 mins, 48 mins, 49 mins, 50 mins, 51 mins, 52 mins, 53 mins, 54 mins, 55 mins, 56 mins, 57 mins, 58 mins, 59 mins, 60 mins, 70 mins, 80 mins, 90 mins, 100 mins, 110 mins, 120 mins, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, or a number or a range between any two of these values after the therapeutically effective amount of the pharmaceutical composition is administered to the subject. For example, therapeutically effective amount of the pharmaceutical composition generates a desired effect in the subject in about 5 minutes to about 100 minutes.

Targeted delivery of psilocybin (or an analogue of psilocybin) using a targeting peptide (e.g., a CNS homing peptide) can shorten delivery time and/or response time. In some embodiments, targeted delivery of psilocybin (or an analogue of psilocybin) using a targeting peptide can shorten delivery time and/or response by, by about, by at least, by at least about, by at most, or by at most about, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or a number or a range between any two of these values.

The therapeutically effective amount of the pharmaceutical composition can generate (or result in) a desired effect in the subject in less time than (or the same amount of time as, or more than than) the time to generate the desired effect when psilocybin (or an analogue of psilocybin) in the pharmaceutical composition is administered to the subject in the absence of the targeting peptide or administered to the subject alone. In some embodiments, the therapeutically effective amount of the pharmaceutical composition generates a desired effect in the subject in 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or a number or a range between any two of these values, of the time needed to generate the desired effect if psilocybin (or an analogue of psilocybin) is administered to the subject in the absence of the targeting peptide or administered to the subject alone. For example, the therapeutically effective amount of the pharmaceutical composition generates a desired effect in the subject 25% to 75% of the time needed to generate the desired effect when psilocybin (or an analogue of psilocybin) is administered to the subject in the absence of the targeting peptide. For example, the therapeutically effective amount of the pharmaceutical composition generates a desired effect in the subject in 25% to 75% of the time needed to generate the desired effect (1) when psilocybin (or an analogue of psilocybin) is administered in the subject and/or (2) when psilocybin (or an analogue of psilocybin) is administered to the subject alone.

The duration of the desired effect is different in different embodiments. In some embodiments, the duration of the desired effect is, is about, is at least, is at least about, is at most, or is at most about, 10 mins, 20 mins, 30 mins, 40 mins, 50 mins, 60 mins, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, 2 days, 3 days, 4 days, 5 days, 6 days, 7 days, or a number or a range between any two of these values. In some embodiments, the desired effect lasts about 1 hour to about 12 hours in the subject.

Pharmacokinetics

Targeted delivery using a targeting peptide (e.g., a CNS homing peptide) can lower the concentration of psilocybin (or an analogue of psilocybin) in the bloodstream of the subject while achieving a desired concentration in cell(s), tissue(s), and/or organ(s) of the target environment affected by the disease, thus reducing undesired side effects which may arise from higher dosage levels. In some embodiments, targeted delivery using a targeting peptide can lower the concentration of psilocybin (or an analogue of psilocybin) in the bloodstream of the subject by, by about, by at least, by at least about, by at most, or by at most about, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or a number or a range between any two of these values, lower.

The maximum concentration (Cₘₐₓ) of psilocybin (or an analogue of psilocybin) in the blood of the subject can be different in different embodiments. In some embodiments, the Cₘₐₓ of psilocybin (or an analogue of psilocybin) in the blood of the subject is, is about, is at least, is at least about, is at most, or is at most about, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 150 ng/ml, 200 ng/ml, 250 ng/ml, 300 ng/ml, 350 ng/ml, 400 ng/ml, 450 ng/ml, 500 ng/ml, 550 ng/ml, 600 ng/ml, 650 ng/ml, 700 ng/ml, 750 ng/ml, 800 ng/ml, 850 ng/ml, 900 ng/ml, 950 ng/ml, 1000 ng/ml, 2 μg/ml, 3 μg/ml, 4 μg/ml, 5 μg/ml, 6 μg/ml, 7 μg/ml, 8 μg/ml, 9 μg/ml, 10 μg/ml, 11 μg/ml, 12 μg/ml, 13 g/ml, 14 μg/ml, 15 μg/ml, 16 μg/ml, 17 μg/ml, 18 μg/ml, 19 μg/ml, 20 μg/ml, 21 μg/ml, 22 g/ml, 23 μg/ml, 24 μg/ml, 25 μg/ml, 26 μg/ml, 27 μg/ml, 28 μg/ml, 29 μg/ml, 30 μg/ml, 31 g/ml, 32 μg/ml, 33 μg/ml, 34 μg/ml, 35 μg/ml, 36 μg/ml, 37 μg/ml, 38 μg/ml, 39 μg/ml, 40 g/ml, 41 μg/ml, 42 μg/ml, 43 μg/ml, 44 μg/ml, 45 μg/ml, 46 μg/ml, 47 μg/ml, 48 μg/ml, 49 g/ml, 50 μg/ml, 60 μg/ml, 70 μg/ml, 80 μg/ml, 90 μg/ml, 100 μg/ml, of a number or a range between any two of these values. For example, the Cₘₐₓ of psilocybin (or an analogue of psilocybin) in the blood of the subject can be about, 2 μg/ml to about 12 μg/ml.

The time to reach the maximum concentration (Tₘₐₓ) of psilocybin (or an analogue of psilocybin) in the blood of the subject can be different in different subjects. In some embodiments, the Tₘₐₓ of psilocybin (or an analogue of psilocybin) in the blood of the subject is, is about, is at least, is at least about, is at most, or is at most about, 1 min, 2 mins, 3 mins, 4 mins, 5 mins, 6 mins, 7 mins, 8 mins, 9 mins, 10 mins, 11 mins, 12 mins, 13 mins, 14 mins, 15 mins, 16 mins, 17 mins, 18 mins, 19 mins, 20 mins, 21 mins, 22 mins, 23 mins, 24 mins, 25 mins, 26 mins, 27 mins, 28 mins, 29 mins, 30 mins, 31 mins, 32 mins, 33 mins, 34 mins, 35 mins, 36 mins, 37 mins, 38 mins, 39 mins, 40 mins, 41 mins, 42 mins, 43 mins, 44 mins, 45 mins, 46 mins, 47 mins, 48 mins, 49 mins, 50 mins, 51 mins, 52 mins, 53 mins, 54 mins, 55 mins, 56 mins, 57 mins, 58 mins, 59 mins, 60 mins, 70 mins, 80 mins, 90 mins, 100 mins, 110 mins, 120 mins, 130 mins, 140 mins, 150 mins, 160 mins, 170 mins, 180 mins, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, of a number or a range between any two of these values. For example, the Tₘₐₓ of psilocybin (or an analogue of psilocybin) in the blood of the subject is about 10 minutes to about 150 minutes.

The elimination half-life (T_{1/2}) of psilocybin (or an analogue of psilocybin) in the blood of the subject can be different in different embodiments. In some embodiments, the elimination half-life of psilocybin (or an analogue of psilocybin) in the blood of the subject is, is about, is at least, is at least about, is at most, or is at most about, 1 min, 2 mins, 3 mins, 4 mins, 5 mins, 6 mins, 7 mins, 8 mins, 9 mins, 10 mins, 11 mins, 12 mins, 13 mins, 14 mins, 15 mins, 16 mins, 17 mins, 18 mins, 19 mins, 20 mins, 21 mins, 22 mins, 23 mins, 24 mins, 25 mins, 26 mins, 27 mins, 28 mins, 29 mins, 30 mins, 31 mins, 32 mins, 33 mins, 34 mins, 35 mins, 36 mins, 37 mins, 38 mins, 39 mins, 40 mins, 41 mins, 42 mins, 43 mins, 44 mins, 45 mins, 46 mins, 47 mins, 48 mins, 49 mins, 50 mins, 51 mins, 52 mins, 53 mins, 54 mins, 55 mins, 56 mins, 57 mins, 58 mins, 59 mins, 60 mins, 70 mins, 80 mins, 90 mins, 100 mins, 110 mins, 120 mins, 130 mins, 140 mins, 150 mins, 160 mins, 170 mins, 180 mins, 190 mins, 200 mins, 210 mins, 220 mins, 230 mins, 240 mins, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, or a number or a range between any two of these values. For example, the elimination half-life of psilocybin (or an analogue of psilocybin) in the blood of the subject can be about 20 minutes to about 200 minutes.

The maximum concentration (Cₘₐₓ) of psilocybin (or an analogue of psilocybin) in cell(s), tissue(s), organ(s), and/or environment(s) thereof of the target environment (e.g., CNS) of the subject can be different in different embodiments. In some embodiments, the Cₘₐₓ of psilocybin (or an analogue of psilocybin) in cell(s), tissue(s), organ(s), and/or environment(s) thereof of the CNS of the subject is, is about, is at least, is at least about, is at most, or is at most about, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 150 ng/ml, 200 ng/ml, 250 ng/ml, 300 ng/ml, 350 ng/ml, 400 ng/ml, 450 ng/ml, 500 ng/ml, 550 ng/ml, 600 ng/ml, 650 ng/ml, 700 ng/ml, 750 ng/ml, 800 ng/ml, 850 ng/ml, 900 ng/ml, 950 ng/ml, 1000 ng/ml, 2 μg/ml, 3 μg/ml, 4 μg/ml, 5 μg/ml, 6 g/ml, 7 μg/ml, 8 μg/ml, 9 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, 20 μg/ml, 21 μg/ml, 22 μg/ml, 23 μg/ml, 24 μg/ml, 25 g/ml, 26 μg/ml, 27 μg/ml, 28 μg/ml, 29 μg/ml, 30 μg/ml, 31 μg/ml, 32 μg/ml, 33 μg/ml, 34 g/ml, 35 μg/ml, 36 μg/ml, 37 μg/ml, 38 μg/ml, 39 μg/ml, 40 μg/ml, 41 μg/ml, 42 μg/ml, 43 g/ml, 44 μg/ml, 45 μg/ml, 46 μg/ml, 47 μg/ml, 48 μg/ml, 49 μg/ml, 50 μg/ml, 60 μg/ml, 70 g/ml, 80 μg/ml, 90 μg/ml, 100 μg/ml, of a number or a range between any two of these values. For example, the Cₘₐₓ of psilocybin (or an analogue of psilocybin) in cell(s), tissue(s), organ(s), and/or environment(s) thereof of the target environment (e.g., CNS) of the subject can be about 2 μg/ml to about 12 μg/ml.

The time to reach the maximum concentration (Tₘₐₓ) of psilocybin (or an analogue of psilocybin) in cell(s), tissue(s), organ(s), and/or environment(s) thereof of the target environment (e.g., CNS) of the subject can be different in different embodiments. In some embodiments, the Tₘₐₓ of psilocybin (or an analogue of psilocybin) in cell(s), tissue(s), organ(s), and/or environment(s) thereof of the target environment (e.g., CNS) of the subject is, is about, is at least, is at least about, is at most, or is at most about, 1 min, 2 mins, 3 mins, 4 mins, 5 mins, 6 mins, 7 mins, 8 mins, 9 mins, 10 mins, 11 mins, 12 mins, 13 mins, 14 mins, 15 mins, 16 mins, 17 mins, 18 mins, 19 mins, 20 mins, 21 mins, 22 mins, 23 mins, 24 mins, 25 mins, 26 mins, 27 mins, 28 mins, 29 mins, 30 mins, 31 mins, 32 mins, 33 mins, 34 mins, 35 mins, 36 mins, 37 mins, 38 mins, 39 mins, 40 mins, 41 mins, 42 mins, 43 mins, 44 mins, 45 mins, 46 mins, 47 mins, 48 mins, 49 mins, 50 mins, 51 mins, 52 mins, 53 mins, 54 mins, 55 mins, 56 mins, 57 mins, 58 mins, 59 mins, 60 mins, 70 mins, 80 mins, 90 mins, 100 mins, 110 mins, 120 mins, 130 mins, 140 mins, 150 mins, 160 mins, 170 mins, 180 mins, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, of a number or a range between any two of these values. For example, the Tₘₐₓ of psilocybin (or an analogue of psilocybin) in cell(s), tissue(s), organ(s), and/or environment(s) thereof of the target environment (e.g., CNS) of the subject can be about 10 minutes to about 150 minutes.

The elimination half-life (T_{1/2}) of psilocybin (or an analogue of psilocybin) in cell(s), tissue(s), organ(s), and/or environment(s) thereof of the target environment (e.g., CNS) of the subject can be different in different embodiments. In some embodiments, the elimination half-life of psilocybin (or an analogue of psilocybin) in cell(s), tissue(s), organ(s), and/or environment(s) thereof of the target environment (e.g., CNS) of the subject is, is about, is at least, is at least about, is at most, or is at most about, 1 min, 2 mins, 3 mins, 4 mins, 5 mins, 6 mins, 7 mins, 8 mins, 9 mins, 10 mins, 11 mins, 12 mins, 13 mins, 14 mins, 15 mins, 16 mins, 17 mins, 18 mins, 19 mins, 20 mins, 21 mins, 22 mins, 23 mins, 24 mins, 25 mins, 26 mins, 27 mins, 28 mins, 29 mins, 30 mins, 31 mins, 32 mins, 33 mins, 34 mins, 35 mins, 36 mins, 37 mins, 38 mins, 39 mins, 40 mins, 41 mins, 42 mins, 43 mins, 44 mins, 45 mins, 46 mins, 47 mins, 48 mins, 49 mins, 50 mins, 51 mins, 52 mins, 53 mins, 54 mins, 55 mins, 56 mins, 57 mins, 58 mins, 59 mins, 60 mins, 70 mins, 80 mins, 90 mins, 100 mins, 110 mins, 120 mins, 130 mins, 140 mins, 150 mins, 160 mins, 170 mins, 180 mins, 190 mins, 200 mins, 210 mins, 220 mins, 230 mins, 240 mins, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, or a number or a range between any two of these values. For example, the elimination half-life of psilocybin (or an analogue of psilocybin) in cell(s), tissue(s), organ(s), and/or environment(s) thereof of the target environment (e.g., CNS) of the subject can be about 20 minutes to about 200 minutes.

The Cₘₐₓ of psilocybin (or an analogue of psilocybin) in cell(s), tissue(s), organ(s), and/or environment(s) thereof of the target environment (e.g., CNS) of the subject can be less than (or the same as, or more than) the Cₘₐₓ of psilocybin (or an analogue of psilocybin) in the blood of the subject. In some embodiments, the Cₘₐₓ of psilocybin (or an analogue of psilocybin) in cell(s), tissue(s), organ(s), and/or environment(s) thereof of the target environment (e.g., CNS) of the subject is, is about, is at least, is at least about, is at most, or is at most about, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 105%, 110%, 115%, 120%, 125%, 130%, 135%, 140%, 145%, 150%, 155%, 160%, 165%, 170%, 175%, 180%, 185%, 190%, 195%, 200%, 210%, 220%, 230%, 240%, 250%, 260%, 270%, 280%, 290%, 300%, or a number or a range between any two of these values, of the Cₘₐₓ of psilocybin (or an analogue of psilocybin) in the blood of the subject. For example, the Cₘₐₓ of psilocybin (or an analogue of psilocybin) in cell(s), tissue(s), organ(s), and/or environment(s) thereof of the target environment (e.g., CNS) of the subject is about 50% to about 150% of the Cₘₐₓ of psilocybin (or an analogue of psilocybin) in the blood of the subject.

The time to reach the maximum concentration (Tₘₐₓ) of psilocybin (or an analogue of psilocybin) in cell(s), tissue(s), organ(s), and/or environment(s) thereof of the target environment (e.g., CNS) of the subject can be less than (or the same as, or more than) the Tₘₐₓ of psilocybin (or an analogue of psilocybin) in the blood of the subject. In some embodiments, the Tₘₐₓ of psilocybin (or an analogue of psilocybin) in cell(s), tissue(s), organ(s), and/or environment(s) thereof of the target environment (e.g., CNS) of the subject is, is about, is at least, is at least about, is at most, or is at most about, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, or a number or a range between any two of these values, of the time to reach the Tₘₐₓ of psilocybin (or an analogue of psilocybin) in the blood of the subject. For example, the Tₘₐₓ of psilocybin (or an analogue of psilocybin) in cell(s), tissue(s), organ(s), and/or environment(s) thereof of the target environment (e.g., CNS) of the subject can be about 100% to about 200% of the time to reach the Tₘₐₓ of psilocybin (or an analogue of psilocybin) in the blood of the subject.

The elimination half-life (T_{1/2}) of psilocybin (or an analogue of psilocybin) in cell(s), tissue(s), organ(s), and/or environment(s) thereof of the target environment (e.g., CNS) of the subject can be less than (or the same as, or more than) the elimination half-life of psilocybin (or an analogue of psilocybin) in the blood of the subject. In some embodiments, the elimination half-life of psilocybin (or an analogue of psilocybin) in cell(s), tissue(s), organ(s), and/or environment(s) thereof of the target environment (e.g., CNS) of the subject is, is about, is at least, is at least about, is at most, or is at most about, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, or a number or a range between any two of these values, of the elimination half-life of psilocybin (or an analogue of psilocybin) in the blood of the subject.

The elimination half-life (T_{1/2}) of psilocybin (or an analogue of psilocybin) in cell(s), tissue(s), organ(s), and/or environment(s) thereof of the target environment (e.g., CNS) of the subject can be less than (or the same as, or more than) the elimination half-life of psilocybin (or an analogue of psilocybin) in the blood of the subject. In some embodiments, the elimination half-life of psilocybin (or an analogue of psilocybin) in cell(s), tissue(s), organ(s), and/or environment(s) thereof of the target environment (e.g., CNS) of the subject is, is about, is at least, is at least about, is at most, or is at most about, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, or a number or a range between any two of these values, of the elimination half-life of psilocybin (or an analogue of psilocybin) in the blood of the subject. For example, the elimination half-life of psilocybin (or an analogue of psilocybin) in cell(s), tissue(s), organ(s), and/or environment(s) thereof of the target environment (e.g., CNS) of the subject is about 100% to about 200% of the elimination half-life of psilocybin (or an analogue of psilocybin) in the blood of the subject.

The maximum concentration (Cₘₐₓ) of psilocybin (or an analogue of psilocybin) in cell(s), tissue(s), organ(s), and/or environment(s) thereof of the target environment (e.g., CNS) of the subject can be less than (or the same as, or more than) the Cₘₐₓ of psilocybin (or an analogue of psilocybin) in cell(s), tissue(s), organ(s), and/or environment(s) thereof of the target environment (e.g., CNS) of the subject when psilocybin (or an analogue of psilocybin) is administered in the absence of the target peptide or administered alone. In some embodiments, the Cₘₐₓ of psilocybin (or an analogue of psilocybin) in cell(s), tissue(s), organ(s), and/or environment(s) thereof of the CNS of the subject is, is about, is at least, is at least about, is at most, or is at most about, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, or a number or a range between any two of these values, of the Cₘₐₓ of psilocybin (or an analogue of psilocybin) in cell(s), tissue(s), organ(s), and/or environment(s) thereof of the target environment (e.g., CNS) of the subject when psilocybin (or an analogue of psilocybin) is administered in the absence of the target peptide or administered alone. For example, the Cₘₐₓ of psilocybin (or an analogue of psilocybin) in cell(s), tissue(s), organ(s), and/or environment(s) thereof of the target environment (e.g., CNS) of the subject is about 50% to about 150% of the Cₘₐₓ of psilocybin (or an analogue of psilocybin) in cell(s), tissue(s), organ(s), and/or environment(s) thereof of the target environment (e.g., CNS) of the subject when psilocybin (or an analogue of psilocybin) is administered in the absence of the CNS homing peptide. For example, the Cₘₐₓ of psilocybin (or an analogue of psilocybin) in cell(s), tissue(s), organ(s), and/or environment(s) thereof of the CNS of the subject is about 50% to about 150% of the Cₘₐₓ of psilocybin (or an analogue of psilocybin) in cell(s), tissue(s), organ(s), and/or environment(s) thereof of the CNS of the subject when psilocybin (or an analogue of psilocybin) is administered alone.

The time to reach the maximum concentration (Tₘₐₓ) of psilocybin (or an analogue of psilocybin) in cell(s), tissue(s), organ(s), and/or environment(s) thereof of the target environment (e.g., CNS) of the subject can be less (or the same as, or more than) the Tₘₐₓ of psilocybin (or an analogue of psilocybin) in cell(s), tissue(s), organ(s), and/or environment(s) thereof of the target environment (e.g., CNS) of the subject when psilocybin (or an analogue of psilocybin) is administered in the absence of the CNS homing peptide or when psilocybin (or an analogue of psilocybin) is administered alone. In some embodiments, the Tₘₐₓ of psilocybin (or an analogue of psilocybin) in cell(s), tissue(s), organ(s), and/or environment(s) thereof of the target environment (e.g., CNS) of the subject is, is about, is at least, is at least about, is at most, or is at most about, 10%, 15%, 20%, 25%, 30%, 35%, 40%, 45%, 50%, 55%, 60%, 65%, 70%, 75%, 80%, 85%, 90%, 95% 100%, 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, or a number or a range between any two of these values, of the Tₘₐₓ of psilocybin (or an analogue of psilocybin) in cell(s), tissue(s), organ(s), and/or environment(s) thereof of the target environment (e.g., CNS) of the subject when psilocybin (or an analogue of psilocybin) is administered in the absence of the CNS homing peptide or when psilocybin (or an analogue of psilocybin) is administered alone. For example, the Tₘₐₓ of psilocybin (or an analogue of psilocybin) in cell(s), tissue(s), organ(s), and/or environment(s) thereof of the target environment (e.g., CNS) of the subject is about 100% to about 200% of the Tₘₐₓ of psilocybin (or an analogue of psilocybin) in cell(s), tissue(s), organ(s), and/or environment(s) thereof of the target environment (e.g., CNS) of the subject when psilocybin (or an analogue of psilocybin) is administered in the absence of the targeting peptide. For example, the Tₘₐₓ of psilocybin (or an analogue of psilocybin) in cell(s), tissue(s), organ(s), and/or environment(s) thereof of the target environment (e.g., CNS) of the subject is about 100% to about 200% of the Tₘₐₓ of psilocybin (or an analogue of psilocybin) in cell(s), tissue(s), organ(s), and/or environment(s) thereof of the target environment (e.g., CNS) of the subject when psilocybin (or an analogue of psilocybin) is administered alone.

For example, the elimination half-life (T_{1/2}) of psilocybin (or an analogue of psilocybin) in cell(s), tissue(s), organ(s), and/or environment(s) thereof of the target environment (e.g., CNS) of the can be less than (or the same as, or more than) the elimination half-life of psilocybin (or an analogue of psilocybin) in cell(s), tissue(s), organ(s), and/or environment(s) thereof of the target environment (e.g., CNS) of the subject when psilocybin (or an analogue of psilocybin) is administered in the absence of the CNS homing peptide or when psilocybin (or an analogue of psilocybin) is administered alone. In some embodiments, the elimination half-life (T_{1/2}) of psilocybin (or an analogue of psilocybin) in cell(s), tissue(s), organ(s), and/or environment(s) thereof of the target environment (e.g., CNS) of the subject is, is about, is at least, is at least about, is at most, or is at most about, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, or a number or a range between any two of these values, of the elimination half-life of psilocybin (or an analogue of psilocybin) in cell(s), tissue(s), organ(s), and/or environment(s) thereof of the target environment (e.g., CNS) of the subject when psilocybin (or an analogue of psilocybin) is administered in the absence of the targeting peptide or when psilocybin (or an analogue of psilocybin) is administered alone. For example, the elimination half-life of psilocybin (or an analogue of psilocybin) in cell(s), tissue(s), organ(s), and/or environment(s) thereof of the target environment (e.g., CNS) of the subject is about 100% to about 200% of the elimination half-life of psilocybin (or an analogue of psilocybin) in cell(s), tissue(s), organ(s), and/or environment(s) thereof of the target environment (e.g., CNS) of the subject when psilocybin (or an analogue of psilocybin) is administered in the absence of the CNS homing peptide. For example the elimination half-life of psilocybin (or an analogue of psilocybin) in cell(s), tissue(s), organ(s), and/or environment(s) thereof of the target environment (e.g., CNS) of the subject is about 100% to about 200% of the elimination half-life of psilocybin (or an analogue of psilocybin) in cell(s), tissue(s), organ(s), and/or environment(s) thereof of the target environment (e.g., CNS) of the subject when psilocybin is administered alone.

### Diseases

The methods, compositions and kits disclosed herein can be used to treat, to prevent, to delay the onset, to slow down the progress, and/or to alleviate one or more symptoms of proliferative diseases such as cancer.

As described herein, the cell(s), tissue(s), and/or organ(s) of the target environment that the targeting peptides disclosed herein are capable of delivering psilocybin or a analogue thereof to can comprise damaged or inflamed cell(s), tissue(s), or organ(s). The target environment can be, for example, nervous system (including CNS). In some embodiments, the cells(s), tissue(s), and/or organ(s) of the target environment comprise the brain, the white matter, the gray matter, the brainstem, the cerebellum, the diencephalon, the cerebrum, the spinal cord, the cranial nerve, cell(s) of any of the preceding, tissue(s) of any of the preceding, or a combination thereof.

A proliferative disease can, for example, be a hyperproliferative disease. In some embodiments, the proliferative disease is cancer. Cancer is an abnormal growth of cells which tend to proliferate in an uncontrolled way and, in some cases, to metastasize (spread). Cancer can involve any tissue of the body and have many different forms in each body area. A tumor can be cancerous or benign. A benign tumor means the tumor can grow but does not spread. A cancerous tumor is malignant, meaning it can grow and spread to other parts of the body. If a cancer spreads (metastasizes), the new tumor bears the same name as the original (primary) tumor.

The methods, compositions and kits disclosed herein can be used to various types of cancer, including but are not limited to, melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer (NSCLC) and small-cell lung cancer (SCLC)), esophageal cancer, squamous cell carcinoma of the head and neck, liver cancer, ovarian cancer, cervical cancer, thyroid cancer, glioblastoma, glioma, leukemia, lymphoma, and other neoplastic malignancies. Additionally, the disease or condition provided herein includes refractory or recurrent malignancies whose growth may be inhibited using the methods and compositions disclosed herein. In some embodiments, the cancer is carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, or a combination thereof. In some embodiments, the cancer is carcinoma, squamous carcinoma (e.g., cervical canal, eyelid, tunica conjunctiva, vagina, lung, oral cavity, skin, urinary bladder, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, the cancer is sarcomata (e.g., myogenic sarcoma), leukosis, neuroma, melanoma, and lymphoma.

The cancer can be a solid tumor, a liquid tumor, or a combination thereof. In some embodiments, the cancer is a solid tumor, including but are not limited to, melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, Merkel cell carcinoma, brain and central nervous system cancers, and any combination thereof. In some embodiments, the cancer is a liquid tumor. In some embodiments, the cancer is a hematological cancer. Non-limiting examples of hematological cancer include Diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), and Multiple myeloma (“MM”).

Non-limiting examples of cancers that can be prevented and/or treated using the methods, compositions and kits disclosed herein include: renal cancer; kidney cancer; glioblastoma multiforme; metastatic breast cancer; breast carcinoma; breast sarcoma; neurofibroma; neurofibromatosis; pediatric tumors; neuroblastoma; malignant melanoma; carcinomas of the epidermis; leukemias such as but not limited to, acute leukemia, acute lymphocytic leukemia, acute myelocytic leukemias such as myeloblastic, promyelocytic, myelomonocytic, monocytic, erythroleukemia leukemias and myclodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone cancer and connective tissue sarcomas such as but not limited to bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangio sarcoma, neurilemmoma, rhabdomyosarcoma, and synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease) and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and ciliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; cervical carcinoma; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; colorectal cancer, KRAS mutated colorectal cancer; colon carcinoma; rectal cancers; liver cancers such as but not limited to hepatocellular carcinoma and hepatoblastoma, gallbladder cancers such as adenocarcinoma; cholangiocarcinomas such as but not limited to papillary, nodular, and diffuse; lung cancers such as KRAS-mutated non-small cell lung cancer, non-small cell lung cancer, squamous cell carcinoma (epidermoid carcinoma), adenocarcinoma, large-cell carcinoma and small-cell lung cancer; lung carcinoma; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, androgen-independent prostate cancer, androgen-dependent prostate cancer, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acrallentiginous melanoma; kidney cancers such as but not limited to renal cell cancer, adenocarcinoma, hypernephroma, fibrosarcoma, transitional cell cancer (renal pelvis and/or uterer); renal carcinoma; Wilms' tumor; and bladder cancers such as but not limited to transitional cell carcinoma, squamous cell cancer, adenocarcinoma, carcinosarcoma. In some embodiments, the cancer is myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, or papillary adenocarcinomas.

### Combination Therapy

The methods, compositions and kits disclosed herein can be used in combination in any cancer treatment methods, agents, or therapies to treat, prevent, inhibit or delay the onset, to slow down the progress of proliferative diseases, and/or to alleviate, prevent or delay the onset of one or more symptoms of proliferative diseases. The proliferative disease is cancer, in some embodiments. For example, the methods disclosed herein can comprise administering to the subject one or more cancer therapies or one or more additional therapeutic agents. Examples of the cancer therapies include, but are not limited to, surgery, chemotherapy, radiation therapy, hormonal therapy, immunotherapy, complementary or alternative therapy, and any combination thereof.

The additional therapeutic agents can comprises one or more chemotherapeutics, including but are not limited to, mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens. Non-limiting examples of the additional therapeutic agents include chemotherapeutic agents, cytotoxic agents, and non-peptide small molecules such as Gleevec® (Imatinib Mesylate), Kyprolis® (carfilzomib), Velcade® (bortezomib), Casodex (bicalutamide), Iressa® (gefitinib), venetoclax, and Adriamycin. Non-limiting examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofiran; spirogermanium; tenuazonic acid; triaziquone; 2,2′,2″ -trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel and docetaxel; retinoic acid; esperamicins; capecitabine; and pharmaceutically acceptable salts, acids or derivatives of any of the above.

In some embodiments, the one or more additional agents comprise anti-hormonal agents capable of regulating or inhibiting hormone action on tumors such as anti-estrogens including for example tamoxifen, (Nolvadex™), raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO).

In some embodiment, the one or more additional therapeutic agents that can be administered to the subject receiving, has received, or will receive, the administration of the compositions disclosed herein comprise currently prescribed anti-cancer drugs such as Herceptin®, Avastin®, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BIBW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, ZD6126, Zosuquidar or a combination thereof.

The methods, compositions and kits disclosed herein can be, in some embodiments, used in combination with radiation therapy for inhibiting abnormal cell growth or treating the proliferative disease such as a hyperproliferative disorder. Non-limiting examples of radiation therapy include, but are not limited to, external-beam therapy, internal radiation therapy, implant radiation, stereotactic radiosurgery, systemic radiation therapy, radiotherapy and permanent or temporary interstitial brachytherapy.

In some embodiments, the methods, compositions or kits disclosed herein is used in combination with one or more of anti-angiogenesis agents, chemotherapeutic agents, anti-neoplastic agents, steroids, signal transduction inhibitors, antiproliferative agents, glycolysis inhibitors, and autophagy inhibitors. The anti-angiogenesis agents can be MMP-2 (matrix-metalloproteinase 2) inhibitors, MMP-9 (matrix-metalloprotienase 9) inhibitors, and COX-11 (cyclooxygenase 11) inhibitors. Anti-angiogenesis agents include, for example, rapamycin, temsirolimus (CCI-779), everolimus (RAD001), sorafenib, sunitinib, and bevacizumab. Non-limiting examples of COX-II inhibitors include alecoxib, valdecoxib, and rofecoxib. Non-limiting examples of anti-neoplastic agents include acemannan, aclarubicin, aldesleukin, alemtuzumab, alitretinoin, altretamine, amifostine, aminolevulinic acid, amrubicin, amsacrine, anagrelide, anastrozole, ANCER, ancestim, ARGLABIN, arsenic trioxide, BAM 002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-N1, interferon alfa-n3, interferon alfacon-1, interferon alpha, natural, interferon beta, interferon beta-1a, interferon beta-1b, interferon gamma, natural interferon gamma-1a, interferon gamma-1b, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, VIRULIZIN, zinostatin stimalamer, or zoledronic acid; abarelix; AE 941 (Aeterna), ambamustine, antisense oligonucleotide, bcl-2 (Genta), APC 8015 (Dendreon), cetuximab, decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techniclone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), or valspodar.

Examples of anti-angiogenic agent include, but are not limited to, ERBITUX™ (IMC-C225), KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF, or soluble VEGF receptors or a ligand binding region thereof) such as AVASTIN™ or VEGF-TRAP™, and anti-VEGF receptor agents (e.g., antibodies or antigen binding regions that specifically bind thereto), EGFR inhibitory agents (e.g., antibodies or antigen binding regions that specifically bind thereto) such as Vectibix (panitumumab), IRESSA™ (gefitinib), TARCEVA™ (erlotinib), anti-Ang1 and anti-Ang2 agents (e.g., antibodies or antigen binding regions specifically binding thereto or to their receptors, e.g., Tie2/Tek), anti-Tie2 kinase inhibitory agents (e.g., antibodies or antigen binding regions that specifically bind thereto), Campath, IL-8, B-FGF, Tek antagonists, anti-TWEAK agents (e.g., specifically binding antibodies or antigen binding regions, or soluble TWEAK receptor antagonists), ADAM distintegrin domain to antagonize the binding of integrin to its ligands, specifically binding anti-eph receptor and/or anti-ephrin antibodies or antigen binding regions, anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto). Autophagy inhibitors include, but are not limited to, chloroquine, 3-methyladenine, hydroxychloroquine (Plaquenil™), bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine.

Non-limiting chemotherapeutic agents include, natural products such as vinca alkaloids (e.g., vinblastine, vincristine, and vinorelbine), paclitaxel, epidipodophyllotoxins (e.g., etoposide and teniposide), antibiotics (e.g., dactinomycin (actinomycin D), daunorubicin, doxorubicin, and idarubicin), anthracyclines, mitoxantrone, bleomycins, plicamycin (mithramycin), mitomycin, enzymes (e.g., L-asparaginase which systemically metabolizes L-asparagine and deprives cells which do not have the capacity to synthesize their own asparagine), antiplatelet agents, antiproliferative/antimitotic alkylating agents such as nitrogen mustards (e.g., mechlorethamine, cyclophosphamide and analogs, melphalan, and chlorambucil), ethylenimines and methylmelamines (e.g., hexaamethylmelaamine and thiotepa), CDK inhibitors (e.g., seliciclib, UCN-01, P1446A-05, PD-0332991, dinaciclib, P27-00, AT-7519, RGB286638, and SCH727965), alkyl sulfonates (e.g., busulfan), nitrosoureas (e.g., carmustine (BCNU) and analogs, and streptozocin), trazenes-dacarbazinine (DTIC), antiproliferative/antimitotic antimetabolites such as folic acid analogs (e.g., methotrexate), pyrimidine analogs (e.g., fluorouracil, floxuridine, and cytarabine), purine analogs and related inhibitors (e.g., mercaptopurine, thioguanine, pentostatin and 2-chlorodeoxyadenosine), aromatase inhibitors (e.g., anastrozole, exemestane, and letrozole), and platinum coordination complexes (e.g., cisplatin and carboplatin), procarbazine, hydroxyurea, mitotane, aminoglutethimide, histone deacetylase (HDAC) inhibitors (e.g., trichostatin, sodium butyrate, apicidan, suberoyl anilide hydroamic acid, vorinostat, LBH 589, romidepsin, ACY-1215, and panobinostat), mTor inhibitors (e.g., temsirolimus, everolimus, ridaforolimus, and sirolimus), KSP(Eg5) inhibitors (e.g., Array 520), DNA binding agents (e.g., Zalypsis), PI3K delta inhibitor (e.g., GS-1101 and TGR-1202), PI3K delta and gamma inhibitor (e.g., CAL-130), multi-kinase inhibitor (e.g., TG02 and sorafenib), hormones (e.g., estrogen) and hormone agonists such as leutinizing hormone releasing hormone (LHRH) agonists (e.g., goserelin, leuprolide and triptorelin), BAFF-neutralizing antibody (e.g., LY2127399), IKK inhibitors, p38MAPK inhibitors, anti-IL-6 (e.g., CNT0328), telomerase inhibitors (e.g., GRN 163L), aurora kinase inhibitors (e.g., MLN8237), cell surface monoclonal antibodies (e.g., anti-CD38 (HUMAX-CD38), anti-CS1 (e.g., elotuzumab), HSP90 inhibitors (e.g., 17 AAG and KOS 953), P13K/Akt inhibitors (e.g., perifosine), Akt inhibitor (e.g., GSK-2141795), PKC inhibitors (e.g., enzastaurin), FTIs (e.g., Zarnestra™), anti-CD138 (e.g., BT062), Torc1/2 specific kinase inhibitor (e.g., INK128), kinase inhibitor (e.g., GS-1101), ER/UPR targeting agent (e.g., MKC-3946), cFMS inhibitor (e.g., ARRY-382), JAK1/2 inhibitor (e.g., CYT387), PARP inhibitor (e.g., olaparib and veliparib (ABT-888)), BCL-2 antagonist. Other chemotherapeutic agents may include mechlorethamine, camptothecin, ifosfamide, tamoxifen, raloxifene, gemcitabine, navelbine, sorafenib, or any analog or derivative variant of the foregoing.

The methods, compositions and kits as disclosed herein, can be used in combination with radiation therapy, hormone therapy, surgery and immunotherapy, which therapies are well known to those skilled in the art.

Non-limiting examples of steroids include 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts and/or derivatives thereof. In a particular embodiment, the compounds of the present invention can also be used in combination with additional pharmaceutically active agents that treat nausea. Examples of agents that can be used to treat nausea include: dronabinol; granisetron; metoclopramide; ondansetron; and prochlorperazine; or a pharmaceutically acceptable salt thereof.

In some embodiments, the one or more additional therapeutic agents that are administered to the subject comprises one or more PD-1 antagonists, PD-L1 antagonists, EGFR inhibitors, MEK inhibitors, PI3K inhibitors, AKT inhibitors, TOR inhibitors, Mcl-1 inhibitors, BCL-2 inhibitors, SHP2 inhibitors, proteasome inhibitors, and immune therapies, including monoclonal antibodies, immunomodulatory imides (IMiDs), anti-PD-1, anti-PDL-1, anti-CTLA4, anti-LAG1, and anti-OX40 agents, GITR agonists, CAR-T cells, and BiTEs. Proteasome inhibitors include, but are not limited to, Kyprolis® (carfilzomib), Velcade® (bortezomib), and oprozomib. Monoclonal antibodies include, but are not limited to, Darzalex® (daratumumab), Herceptin® (trastuzumab), Avastin® (bevacizumab), Rituxan® (rituximab), Lucentis® (ranibizumab), and Eylea® (aflibercept).

### Formulation and Administration

Some embodiments provided herein are directed to an effective amount of a pharmaceutical composition comprising a compound (e.g., psilocybin associated with a targeting peptide (for example a CNS homing peptide)) and at least one pharmaceutically acceptable carrier or excipient. Some embodiments provided herein are directed to a pharmaceutical composition comprising an effective amount of a compound (e.g., psilocybin associated with a targeting peptide) and at least one pharmaceutically acceptable carrier or excipient.

Carriers or excipients can be used to produce compositions. The carriers or excipients can be chosen to facilitate administration of the compound or composition. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

Suitable dosage forms, in part, depend upon the use or the route of administration, for example, oral, transdermal, transmucosal, inhalant, or by injection (parenteral). Such dosage forms should allow the compound to reach target cells. Other factors include considerations such as toxicity and dosage forms that retard the compound or composition from exerting its effects.

The compound or composition can be administered by different routes including intravenous, intraperitoneal, subcutaneous, intramuscular, oral, transmucosal, rectal, transdermal, or inhalant. In some embodiments, the composition can be administered by oral administration. For oral administration, for example, the compound or composition can be formulated into conventional oral dosage forms such as capsules, tablets, and liquid preparations such as syrups, elixirs, and concentrated drops.

For inhalants, the compound or composition may be formulated as dry powder or a suitable solution, suspension, or aerosol. Powders and solutions may be formulated with suitable additives. For example, powders may include a suitable powder base such as lactose or starch, and solutions may comprise propylene glycol, sterile water, ethanol, sodium chloride and other additives, such as acid, alkali and buffer salts. Such solutions or suspensions may be administered by inhaling via spray, pump, atomizer, or nebulizer, and the like. The compound or composition may also be used in combination with other inhaled therapies.

Pharmaceutical preparations for oral use can be obtained, for example, by combining the compound with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethylcellulose (CMC), and/or polyvinylpyrrolidone (PVP, povidone). If desired, disintegrating agents may be added, such as the cross-linked polyvinylpyrrolidone, agar, or alginic acid, or a salt thereof such as sodium alginate.

Dragee cores are provided with suitable coatings. For this purpose, concentrated sugar solutions may be used, which may optionally contain, for example, gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin (“gelcaps”), as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the compound or composition may be dissolved or suspended in suitable liquids, such as fatty oils, liquid paraffin, or liquid polyethylene glycols (PEGs). In addition, stabilizers may be added.

Alternatively, injection (parenteral administration) may be used, e.g., intramuscular, intravenous, intraperitoneal, and/or subcutaneous. For injection, the compound or composition can be formulated in sterile liquid solutions, such as in physiologically compatible buffers or solutions, such as saline solution, Hank's solution, or Ringer's solution. In addition, the compound or composition may be formulated in solid form and redissolved or suspended immediately prior to use. Lyophilized forms can also be produced.

Administration can also be by transmucosal, topical, transdermal, or inhalant means. For transmucosal, topical or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays or suppositories (rectal or vaginal).

The topical compositions of this disclosure are formulated as oils, creams, lotions, ointments, and the like by choice of appropriate carriers known in the art. Suitable carriers include vegetable or mineral oils, white petrolatum (white soft paraffin), branched chain fats or oils, animal fats and high molecular weight alcohol (greater than C₁₂). In another embodiment, the carriers are those in which the active ingredient is soluble. Emulsifiers, stabilizers, humectants and antioxidants may also be included as well as agents imparting color or fragrance, if desired. Creams for topical application are formulated from a mixture of mineral oil, self-emulsifying beeswax and water in which mixture the active ingredient, dissolved in a small amount solvent (e.g. an oil), is admixed. Additionally, administration by transdermal means may comprise a transdermal patch or dressing such as a bandage impregnated with an active ingredient and optionally one or more carriers or diluents known in the art. To be administered in the form of a transdermal delivery system, the dosage administration will, of course, be continuous rather than intermittent throughout the dosage regimen.

In some embodiments, the present disclosure provides kits that include a compound or a pharmaceutical composition thereof. In some embodiments, the compound or composition is packaged, e.g., in a vial, bottle, flask, which may be further packaged, e.g., within a box, envelope, or bag; the compound or composition is approved by the U.S. Food and Drug Administration or similar regulatory agency for administration to a mammal, e.g., a human; the compound or composition is approved for administration to a mammal, e.g., a human, for a proliferative disease; the kits described herein may include written instructions for use and/or other indication that the compound or composition is suitable or approved for administration to a mammal, e.g., a human, for a disease or condition as described herein; and the compound or composition may be packaged in unit dose or single dose form, e.g., single dose pills, capsules, or the like.

The compounds described herein can be used in combination with the agents disclosed herein or other suitable agents, depending on the condition being treated. Hence, in some embodiments the one or more compounds of the disclosure will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound described herein and any of the agents described above can be formulated together in the same dosage form and administered simultaneously. Alternatively, a compound of the disclosure and any of the agents described above can be simultaneously administered, wherein both the agents are present in separate formulations. In some embodiments, a compound of the present disclosure can be administered just followed by and any of the agents described above, or vice versa. In some embodiments of the separate administration protocol, a compound of the disclosure and any of the agents described above are administered a few minutes apart, or a few hours apart, or a few days apart.

### EXAMPLES

Some aspects of the embodiments discussed above are disclosed in further detail in the following examples, which are not in any way intended to limit the scope of the present disclosure.

### Example 1

### Peptide-Guided Therapy Using Psilocybin

In this example, the pharmacokinetics of psilocybin delivered to rats having central nervous system (CNS) disease via liposomes are evaluated. Psilocybin is delivered herein via peptide-guided liposomes, which is for enhancing the efficacy and/or toxicity profile of psilocybin. As disclosed herein, the peptide-guided liposome delivery can also be adapted for use with other drugs because liposomes can be loaded with both hydrophobic and hydrophilic drugs, thus, permitting combination drug therapy.

Multiple sclerosis (MS) is an autoimmune disease affecting the CNS. Inflammation and demyelination of neurons resulting from a self-directed immune attack leads to muscle weakness and paralysis. Despite the availability of several drugs, many MS patients do not respond well to them. In addition, these drugs have many side effects. Thus, there is an urgent need to develop more efficient drug delivery approaches with the purpose of increasing efficacy but reducing adverse effects of these drugs. The currently used drugs are taken orally or given by injection. Therefore, several organs become unintended targets of those drugs that are primarily intended to target the diseased tissue in the CNS. Targeted drug delivery to the CNS can overcome many of the limitations of current therapies. One of the CNS-homing peptides disclosed herein is used as a guide to direct one or more drugs into the CNS and thereby minimize or eliminate off-target side effects. Psilocybin is incorporated within a nanoparticle, which displays one of the CNS-homing peptide disclosed herein on its surface. The nanoparticle has polyethylene glycol (PEG) on its surface to minimize the otherwise rapid clearance of the particles by the reticuloendothelial system.

Animal Model with Experimental Autoimmune Encephalomyelitis (EAE) for Human MS

Relapsing-remitting disease model in SJL (H-2s) mice (SJL mice), which closely mimics the relapsing nature of MS in humans, is used in this study. In this animal model, proteolipid protein (PLP), a neuronal antigen, is used for disease induction. PLP emulsified in complete Freund's adjuvant (CFA) is injected subcutaneously (s.c.). The onset of signs of EAE and disease progression, including relapses and remissions, are graded on a standardized disease severity scale. Changes in clinical disease are then confirmed by histopathological signs of demyelination in the brain and spinal cord.

### Objective of the Study

Using the EAE model, it is expected that CNS targeting peptide-displaying liposomes can effectively deliver psilocybin to the CNS, and it is superior to plain liposomes or free drug in inhibiting the relapses and progression of EAE. Additionally, specific cell types within the CNS that are targeted by the CNS-homing peptide and CNS-homing peptide-displaying liposomes are expected to be identified.

The CNS-homing peptide-guided drug delivery system is tested for its relative efficacy against relapsing disease in SJL mice compared with that of drug delivery via plain liposomes or as unpackaged (free) drug. To monitor various parameters of systemic toxicity (e.g., toxicity to the liver, kidney, heart, and muscle) in experimental and control groups, the efficacy versus safety profiles of the drug administered via 3 different modalities are compared. The cell types within CNS (e.g., neurons, astrocytes, glial cells, endothelial cells) that are targeted by the CNS homing peptide and CNS homing peptide-displaying liposomes are defined to determine how and where drug-carrying liposomes interact with the CNS tissue cells.

### Experimental Plan

Experiment 1: Pre-clinical efficacy testing in EAE mice. A cohort of SJL mice is injected s.c. with PLP for EAE induction, which appears about d 10 after PLP injection. At the time of onset of EAE, mice are be randomized into 3 sets of two groups each for treatment with psilocybin administered in three different formulations. A group of untreated EAE mice is served as another control.
(a) Set 1: Peptide-guided liposomal psilocybin delivery. Psilocybin (Group 1) are entrapped in liposomes. Liposomes containing vehicle only (Group 2) is served as a control. All these liposomes display on their cell surface the CNS-homing peptide as well as polyethylene glycol (PEG); the latter to reduce their clearance by the reticuloendothelial system.
(b) Set 2: liposomes lacking the CNS-homing peptide. Plain liposomes containing psilocybin (Group 3) or vehicle (Group 4) are prepared.
(c) Set 3: plain psilocybin (Group 5) or vehicle (Group 6). Set-3 is served as controls for the other two sets.

The liposomes or free drug/vehicles are injected intravenously (iv) into EAE mice. One injection is given at the time of EAE onset, with a second injection after 3 days, and a third injection after another 3 days, coinciding with the peak phase of disease (about d 16 after PLP injection). Throughout this period, the EAE severity of mice is graded in a blinded fashion.

Experiment 2: Histopathological and serological confirmation of disease severity and/or toxicity. After 7-10 days of the third injection, all mice are sacrificed, and various tissues and sera collected are tested as follows: (i) Efficacy: the CNS tissue (brain and spinal cord) are histologically analyzed for signs of perivascular inflammation and demyelination for confirmation of clinical observations; and (ii) Toxicity: the sera are tested for tissue enzymes/markers for toxicity to liver, kidney, pancreas, etc.

### TERMINOLOGY

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of treating a proliferative disease in a subject in need thereof, comprising:
administering to a subject a therapeutically effective amount of a pharmaceutical composition, wherein the pharmaceutical composition comprises a targeting peptide comprising an amino acid sequence of any one of SEQ ID NOs: 1-22 associated with psilocybin, or a pharmaceutically acceptable salt, co-crystal, polymorph, hydrate, solvate, stereoisomer, or pro-drug of psilocybin, and wherein the targeting peptide is capable of delivering the psilocybin, or the pharmaceutically acceptable salt, co-crystal, polymorph, hydrate, solvate, stereoisomer, or pro-drug of psilocybin, to a target environment of the subject.

2. A method of alleviating one or more symptoms of a proliferative disease, or preventing or delaying the onset of one or more symptoms of a proliferative disease, in a subject in need thereof, comprising:
administering to a subject a therapeutically effective amount of a pharmaceutical composition, wherein the pharmaceutical composition comprises a targeting peptide comprising an amino acid sequence of any one of SEQ ID NOs: 1-22 associated with psilocybin, or a pharmaceutically acceptable salt, co-crystal, polymorph, hydrate, solvate, stereoisomer, or pro-drug of psilocybin, and wherein the targeting peptide is capable of delivering the psilocybin, or the pharmaceutically acceptable salt, co-crystal, polymorph, hydrate, solvate, stereoisomer, or pro-drug of psilocybin, to a target environment of the subject.

3. The method of claim 2, wherein the one or more symptoms comprise pain.

4. The method of claim 1, wherein the proliferative disease is cancer.

5. The method of claim 1, wherein the targeting peptide is a central nervous system (CNS) targeting peptide and/or wherein the target environment is the nervous system.

6. The method of claim 1, wherein the targeting peptide is directly associated with psilocybin, or a pharmaceutically acceptable salt, co-crystal, polymorph, hydrate, solvate, stereoisomer, or pro-drug of psilocybin.

7. The method of claim 6, wherein the targeting peptide is covalently attached with psilocybin, or a pharmaceutically acceptable salt, co-crystal, polymorph, hydrate, solvate, stereoisomer, or pro-drug of psilocybin.

8. The method of claim 6, wherein the targeting peptide is non-covalently attached with psilocybin, or a pharmaceutically acceptable salt, co-crystal, polymorph, hydrate, solvate, stereoisomer, or pro-drug of psilocybin.

9. The method of claim 1, wherein the targeting peptide is indirectly associated with psilocybin, or a pharmaceutically acceptable salt, co-crystal, polymorph, hydrate, solvate, stereoisomer, or pro-drug of psilocybin.

10.-12. (canceled)

13. The method of claim 1, wherein the therapeutically effective amount of the pharmaceutical composition comprises about 1 mg to about 100 mg of psilocybin, or a pharmaceutically acceptable salt, co-crystal, polymorph, hydrate, solvate, stereoisomer, or pro-drug of psilocybin, and/or wherein the therapeutically effective amount comprises about 1 mg to about 100 mg of the pharmaceutical composition.

14.-23. (canceled)

24. The method of claim 1, wherein the administering comprises administering to the subject the therapeutically effective amount of the pharmaceutical composition orally, intravenously, or a combination thereof.

25.-27. (canceled)

28. A pharmaceutical composition for use in the treatment of a proliferative disease comprising a targeting peptide associated with psilocybin, or a pharmaceutically acceptable salt, co-crystal, polymorph, hydrate, solvate, stereoisomer, or pro-drug thereof, wherein the targeting peptide comprises an amino acid sequence of any one of SEQ ID NOs: 1-22, and wherein the targeting peptide is capable of delivering the psilocybin, or the pharmaceutically acceptable salt, co-crystal, polymorph, hydrate, solvate, stereoisomer, or pro-drug of psilocybin, to a target environment of a subject.

29.-30. (canceled)

31. The pharmaceutical composition of claim 28, wherein the proliferative disease is cancer.

32. The pharmaceutical composition of claim 28, wherein the targeting peptide is a central nervous system (CNS) targeting peptide and/or wherein the target environment is the nervous system.

33. The pharmaceutical composition of claim 28, wherein the targeting peptide is directly associated with psilocybin, or a pharmaceutically acceptable salt, co-crystal, polymorph, hydrate, solvate, stereoisomer, or pro-drug of psilocybin.

34. The pharmaceutical composition of claim 33, wherein the targeting peptide is covalently attached with psilocybin, or a pharmaceutically acceptable salt, co-crystal, polymorph, hydrate, solvate, stereoisomer, or pro-drug of psilocybin.

35. The pharmaceutical composition of claim 33, wherein the targeting peptide is non-covalently attached with psilocybin, or a pharmaceutically acceptable salt, co-crystal, polymorph, hydrate, solvate, stereoisomer, or pro-drug of psilocybin.

36. (canceled)

37. The pharmaceutical composition of claim 28, wherein the pharmaceutical composition comprises a delivery vehicle comprising the targeting peptide on an outer surface of the delivery vehicle.

38.-54. (canceled)

55. The method of claim 4, wherein the cancer is carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, or a combination thereof; (2) wherein the disease is a solid tumor.

56. The pharmaceutical composition of claim 31, wherein the cancer is carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, or a combination thereof; (2) wherein the disease is a solid tumor.
